(19) United States
(12) Patent Application Publication
(10) Pub. No.: US 2006/0205996 A1
(43) Pub. Date: Sep. 14, 2006

(54) Incontinence treatment with urethral guide

(75) Inventors: James B. Presthus, Edina, MN (US); Timothy G. Dietz, Califon, NJ (US); Stanley Levy JR., Saratoga, CA (US); F. Allen House, Pleasanton, CA (US); Steven H. Trebotich, Newark, CA (US); Abdul M. Tayeb, San Leandro, CA (US); Oren A. Mosher, Castro Valley, CA (US); George L. Matlock, Pleasanton, CA (US); Terry E. Spraker, Portola Valley, CA (US); Daniel D. Merrick, Dublin, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Solorant Medical, Inc., Livermore, CA (US)

(21) Appl. No.: 11/368,277

(22) Filed: Mar. 2, 2006

Related U.S. Application Data
(63) Continuation-in-part of application No. 10/301,561, filed on Nov. 20, 2002, now Pat. No. 7,052,453.
Application No. 10/301,561 is a continuation-in-part of application No. 09/991,368, filed on Nov. 20, 2001, now Pat. No. 6,685,623.

(51) Int. Cl. A61F 2/00

(52) U.S. Cl. 600/29

(57) ABSTRACT

Incontinence systems and methods for directing treatment to a target tissue of a patient comprise a guide having a first palpation member and a probe body having a treatment zone and a second palpation member. The guide is configured to be inserted into a urethra of the patient. The first palpation member is positioned in a fixed relationship to an anatomical landmark, such as a bladder neck. The probe body is configured to be inserted into a vagina of the patient. The second palpation member is registered proximal the first palpation member so as to position the treatment zone of the probe adjacent the target tissue of the patient and away from the nerves and/or tissues in the area of the bladder neck and bladder.

### CROSS-REFERENCE TO RELATED APPLICATIONS

This application is a continuation-in-part of U.S. patent application Ser. No. 10/301,561, filed Nov. 20, 2002, which is a continuation-in-part of U.S. patent application Ser. No. 09/991,368, filed Nov. 20, 2001, now U.S. Pat. No. 6,685,623, the complete disclosures of which are incorporated herein by reference.

### BACKGROUND OF THE INVENTION

The present invention relates generally to medical devices methods, systems, and kits. More specifically, the present invention provides devices and methods for positioning a treatment surface adjacent a target tissue to selectively heat and shrink tissues, particularly for the noninvasive treatment of urinary incontinence, hernias, cosmetic surgery, and the like.

Urinary incontinence arises in both women and men with varying degrees of severity, and from different causes. In men, the condition occurs almost exclusively as a result of prostatectomies which result in mechanical damage to the sphincter. In women, the condition typically arises after pregnancy where musculoskeletal damage has occurred as a result of inelastic stretching of the structures which support the genitourinary tract. Specifically, pregnancy can result in inelastic stretching of the pelvic floor, the external vaginal sphincter, and most often, the tissue structures which support the bladder and bladder neck region. In each of these cases, urinary leakage typically occurs when a patient's intra-abdominal pressure increases as a result of stress, e.g. coughing, sneezing, laughing, exercise, or the like.

Treatment of urinary incontinence can take a variety of forms. Most simply, the patient can wear absorptive devices or clothing, which is often sufficient for minor leakage events. Alternatively or additionally, patients may undertake exercises intended to strengthen the muscles in the pelvic region, or may attempt behavior modification intended to reduce the incidence of urinary leakage.

In cases where such noninterventional approaches are inadequate or unacceptable, the patient may undergo surgery to correct the problem. A variety of procedures have been developed to correct urinary incontinence in women. Several of these procedures are specifically intended to support the bladder neck region. For example, sutures, straps, or other artificial structures are often looped around the bladder neck and affixed to the pelvis, the endopelvic fascia, the ligaments which support the bladder, or the like. Other procedures involve surgical injections of bulking agents, inflatable balloons, or other elements to mechanically support the bladder neck.

It has recently been proposed to selectively deliver RF energy to gently heat fascia and other collagenated support tissues to treat incontinence. One problem associated with delivering RF energy to the targeted tissue is the alignment of the electrodes with the target tissue. Direct heating of target tissue is often complicated since the target tissue is offset laterally and separated from the urethra by triangular shaped fascia sheets supporting the urethra. These urethra supporting fascia sheets often contain nerve bundles and other structure that would not benefit from heating. In fact, injury to these nerve bundles may even promote incontinence, instead of providing relief from incontinence.

For these reasons, it would be desirable to provide improved devices, methods, systems, and kits for providing improved alignment devices and methods that would improve the positioning of heating electrodes adjacent the target tissue and away from the surrounding, sensitive nerve bundles.

### BRIEF SUMMARY OF THE INVENTION

The present invention provides devices, methods, systems, and kits for positioning a treatment surface adjacent a target tissue. In one embodiment, the present invention can be used for treating urinary incontinence.

Embodiments of the probe and guide of the present invention can accurately position a treatment surface, such as an electrode array, adjacent a target tissue by utilizing the human anatomy to help guide the treatment surface into contact with the target tissue. Generally, the guide can be inserted into a first body orifice and the probe can be inserted into a second body orifice and placed in a predetermined position relative to the guide so as to position the treatment surface adjacent the target tissue in the second body orifice.

In some embodiments, the guide can be inserted into the urethra to help position the treatment surface adjacent the target tissue in the vagina. In the embodiments, the probes can include a probe body comprising a treatment surface. A probe body can be registered with the guide that is positioned in the urethra and positionable in the vagina to help align the treatment surface with a target tissue in the vagina.

In one embodiment, the urethral guide can be physically couplable to the probe body. Optionally, the urethral guide can be removably attached to the probe body and/or rotatably attached to the probe body. The rotatable attachment can provide flexibility in positioning treatment surface adjacent the target tissue. The removable attachment allows the probe body and urethral guide to be independently inserted into the body orifices. After both have been inserted, the two can optionally be attached to align the treatment assembly with the target tissue. Optionally, the probes of the present invention may have a coupling structure on each side of the probe body to provide proper alignment of the treatment surface with target tissue both to the left and right of the non-target urethra tissue.

Some embodiments of the urethral guides of the present invention can be configured to bias the electrodes into the target tissue. Such biasing can improve the efficiency of electrical energy delivery to the target tissue while avoiding energy delivery to the surrounding non-target tissue if the electrodes are not in proper contact with the target tissue.

Some embodiments of the probe body and guide means can be rigid and rigidly connected to each other. The rigid configuration of the probes of the present invention allows the physician to maintain the position of the treatment surface relative to the target tissue. Other embodiments of the probe body and guide, however, can be partly or completely flexible.

In other embodiments, the urethral guide will not be physically coupled to the probe body but will be registered with the probe body through its position relative to the position of the probe body.

In one embodiment, the urethral guide can be registered with or in communication with the probe body based on its physical location relative to the probe body. A palpation member (such as a bump or indentation, landmark, a clip, a marking, or the like) on the urethral guide and the probe body can provide landmarks for the physician to assist the physician in positioning the treatment surface of the probe body adjacent the target tissue.

In another embodiment, the urethral guide can be registered with the probe body through an electromagnetic coupling such as a Radiofrequency (RF) coupling, magnetic coupling, or light sensing coupling (either visible or infrared). In such embodiments, the urethral guide and probe body do not have to be physically coupled with each other (but can be, if desired) and typically can be moved freely, relative to each other.

In one embodiment, the urethral guide and/or the probe body can include one or more RF transmitter(s) and RF sensor(s). The RF coupling can provide a RF position signal to a controller that is indicative of the spacing between the sensors and transmitters on the urethral guide and the probe. The RF signal can be delivered to the controller so that the controller can inform the user of the positioning of the probe body relative to the urethral guide. Once the urethral guide and probe have been placed in their proper positions in the body orifices and in a proper, predetermined position relative to each other, the RF sensor will produce a position signal that informs the controller that the probe is disposed in a position that places the treatment surface adjacent the target tissue.

In another embodiment, a magnetic coupling that includes one or more magnetic field transmitter(s) (e.g., an electromagnet) and/or one or more magnetic field sensors (e.g., Hall Effect sensors) to position the probe body in a proper position relative to the urethral guide. The magnetic coupling can provide an electromagnetic signal that is indicative of the spacing between the urethral guide and the probe. The magnetic field signal can be delivered to the controller through the magnetic field sensors so that the controller can inform the user of the positioning of the probe body. Once the urethral guide and probe have been placed in their proper position in the body orifices and in a proper, predetermined position relative to each other, the magnetic field sensor will produce a signal that indicates a proper positioning of the probe relative to the urethral guide.

In some configurations, the controller can be configured to inform the user that there is an improper or proper spacing between the probe body and urethral guide. In some configurations, the controller can be configured to prevent delivery of energy to the treatment surface until a proper spacing or proper positioning of the treatment surface is achieved. In other configurations, the controller can be configured to provide an indication (such as a readout on a monitor, or an audible signal) that there is a proper positioning of the probe body in the vagina relative to the urethral guide.

The guides of the present invention can also optionally include an expansible member adjacent its distal end. The urethral guide can be moved through the urethra and into the patient's bladder. Once in the bladder, the expansible member can be expanded so as to prevent proximal movement of the urethral guide and probe body.

In some embodiments, the urethral guide can include a temperature sensor that is coupled to the controller to allow the user to monitor the tissue temperature of the urethra.

The methods of the present invention generally comprise positioning a guide in the patient's body and guiding a treatment surface, such as an electrode array to a target tissue. Once the treatment surface is positioned against the target tissue, the target tissue can be treated. In some embodiments, treatments comprise delivering an electrical energy to heat and shrink or stiffen the target tissue.

One embodiment of the method of the present invention comprises placing a guide into a first body orifice (e.g., urethra). A treatment probe having a treatment surface can be inserted into a second body orifice (e.g., vagina). The probe can be placed in a predetermined position relative to the guide (e.g., registered) so as to position the treatment surface in proper alignment with a target tissue in the second body orifice. Thereafter, the target tissue can be treated with the treatment surface

In some embodiments, the methods of the present invention can include the step of measuring the length of the patient's urethra. Once the patient's urethra has been measured, the physician can then calculate a predetermined distance of the urethra for advancement of the urethral guide. In one embodiment, the predetermined distance is approximately a mid-urethra point. In other embodiments, however, the predetermined target distance can be other target distances, that are larger or smaller than the mid-urethra point. Locating the midpoint of the urethra can be done automatically or the process of midpoint location can be carried out by manually measuring the length of the patient's urethra and inserting marked positioning devices to a position called for by the measured urethral length.

Once the mid-urethra point is calculated (or other predetermined distance), the urethral guide can be placed in the urethra and advanced to the mid-urethra point to “mark” the mid-urethra. In some embodiments, the mid-urethra point can be marked with the urethral guide by using an RF transmitter, magnetic field transmitter, or a mechanical palpation member that can indicate to the physician the position of the mid-urethra. Once the mid-urethra point is marked, a variety of methods can be used to position the treatment surface near the marker and adjacent the target tissue. Thereafter, the treatment surface can be used to treat the target tissue.

The present invention further provides kits for treating incontinence. The kits of the present invention typically include any of the probes and guides as described herein. The kits will generally include a package for holding the probe, guide, and instructions for use which describe any of the exemplary methods described herein. Optionally, the kits may include a controller, power source, electrical connections, or the like.

A further understanding of the nature and advantages of the invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1A illustrates an embodiment of an electrosurgical probe of the present invention;

FIG. 1B is a close up perspective view of an exemplary coupling assembly;

FIG. 2 illustrates an embodiment of an urethral guide shaft of the present invention;

FIG. 3 is a simplified end view of a distal orifice and expansible member disposed on guide shaft;

FIG. 4 is a simplified side view of an embodiment of the expansible member;

FIG. 5 is a simplified view of an alternative embodiment of the noninvasive probe of the present invention;

FIG. 6 illustrates an exemplary embodiment of a coupling structure on two sides of the probe body which allows for positioning of the probe body against target tissue on both the left and right side of the urethra;

FIG. 7 is a simplified cross sectional view of a radiused electrode and a guide of the present invention illustrating a lateral offset of the guide relative to the probe body and an orthogonal offset relative to a plane of the electrode;

FIG. 8 is a simplified cross sectional front view of target tissue of an exemplary method of the present invention;

FIG. 9 is a cross sectional view of the tissue that can be targeted for non-invasive treatment using the methods of the present invention;

FIGS. 9A-9C illustrate some embodiments that comprise a urethral guide that is rotatably attached to the probe body about at least one axis;

FIG. 10 illustrates placement of an embodiment of the guide into the urethra;

FIG. 11 illustrates expanding of the expansible member in the bladder;

FIG. 12 illustrates placement of the probe into the vagina;

FIG. 13 illustrates coupling of the guide to the probe body in an offset configuration and treating the target tissue;

FIG. 14 illustrates an embodiment that includes a mechanical palpation member coupled to the urethral guide to indicate a mid-urethra point;

FIG. 15 illustrates the urethral guide of FIG. 14 with a probe;

FIG. 16 illustrates yet another embodiment of an urethral guide of the present invention that includes an expansion member;

FIG. 17 illustrates the urethral guide of FIG. 16 and a probe of the present invention;

FIGS. 18A and 18B are cross sectional views of a simplified urethral guide having an expandable portion;

FIGS. 19A and 19B illustrate an embodiment that includes RF coupling;

FIGS. 20A and 20B illustrate an embodiment that include a magnetic coupling;

FIG. 21 schematically illustrates a CPU of a controller coupled to an output display that shows a graphic representation of the urethral guide and probe;

FIG. 22 schematically illustrates a simplified method of the present invention;

FIGS. 23A to 23F illustrate one embodiment of a method and device for measuring a length and a mid-urethral length;

FIGS. 24A to 24C illustrates another embodiment of a method and device for automatically locating the mid-urethral position and placing a sensor or other position indicating device at the mid-urethra;

FIG. 25 illustrates an embodiment of a kit of the present invention;

FIGS. 26A to 26D illustrate an exemplary embodiment of the urethral measuring positioning applicator;

FIGS. 27A and 27B illustrate an exemplary embodiment of the probe; and

FIGS. 28A to 28G illustrate an exemplary palpation method for positioning the system of FIGS. 26 and 27 within a patient's body.

### DESCRIPTION OF THE SPECIFIC EMBODIMENTS

The present invention provides methods, devices, systems, and kits for accurately positioning a treatment surface, such as an electrode array, adjacent fascia and other collagenated tissues to selectively treat the target tissue. In a particular embodiment, the present invention accurately directs an electrical current flux through the target tissue between bipolar electrodes that are contacting the target tissue to shrink or stiffen the collagenated tissue.

Exemplary embodiments of the present invention heat target tissue in the vagina for treating urinary incontinence. The urethra is composed of muscle structures that allow it to function as a sphincter controlling the release of urine from the bladder. These muscles are controlled by nerve bundles that in part run in close proximity to the urethra-bladder junction and along the axis of the urethra. Pelvic surgery in this region has been associated with the development of intrinsic sphincter deficiency of the urethra. It is therefore important that any tissue treatment avoid areas containing nerve pathways that supply the urethra. Because the present invention provides accurate placement with the target tissue, collateral damage to surrounding nerve bundles and other organs can be reduced.

While the remaining discussion will be directed at treating incontinence in a female patient, it should be appreciated that the concepts of the present invention are further applicable to other noninvasive and invasive surgical procedures, and are not limited to treating urinary incontinence.

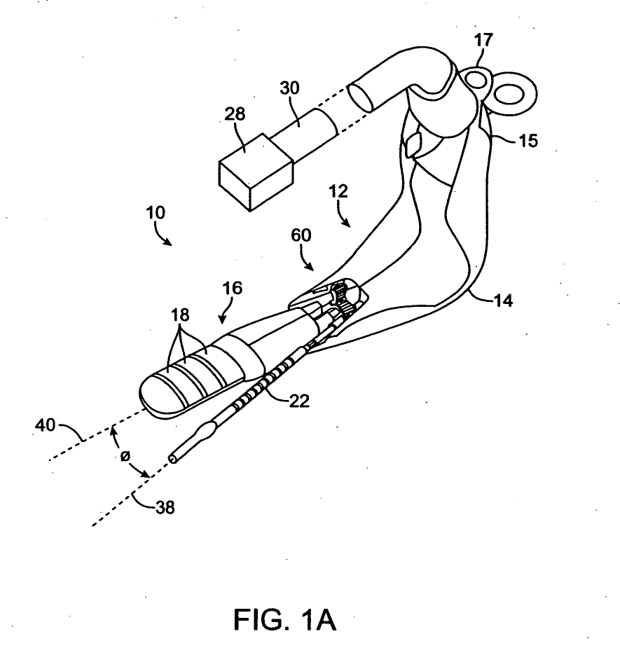

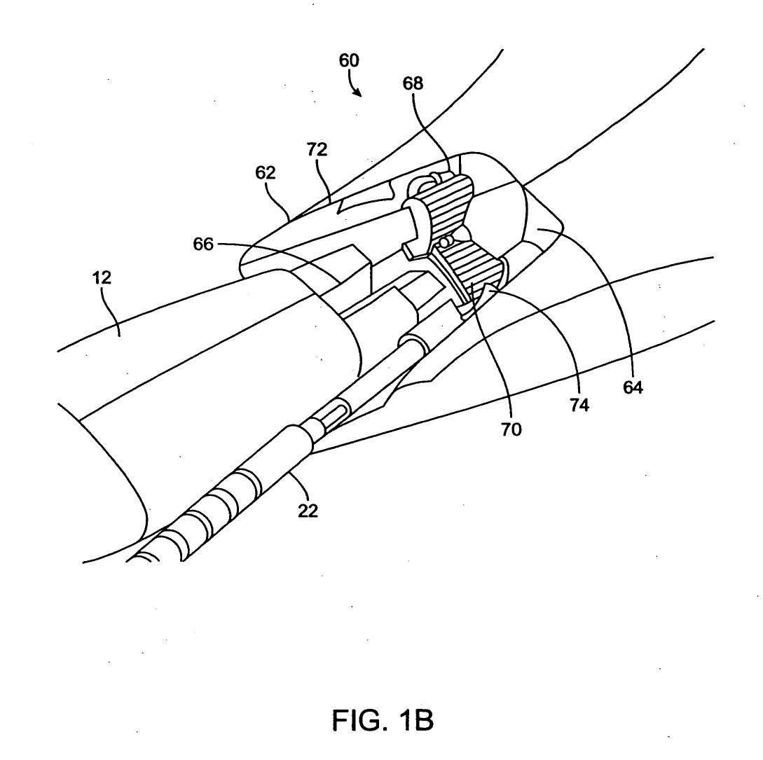

FIG. 1A illustrates an exemplary electrosurgical probe 10 of the present invention. The electrosurgical probe includes an applicator or probe body 12 having a proximal portion 14 and a distal portion 16. Proximal portion 14 of the probe body 12 generally includes a handle 15 and one or more triggers or switches 17 for activating a delivery of electrical energy to the target tissue or for deploying a temperature probe into the target tissue to monitor the tissue temperature during treatment. Distal portion 16 can include a treatment surface 18 that has at least one electrode or other type of treatment assembly. The treatment assembly can include an electrode on a needle, ultrasound transducer, microwave antenna, a needle for delivery of a therapeutic agent, or the like. A guide body or shaft 22 can be attachable to the probe body 12 to assist in the proper positioning of the distal portion 16 of probe body 12 and treatment surface 18 with a target tissue. As will be described in detail below, other embodiments include a guide 22 that is not attached to probe body 12.

Systems of the present invention can further include a power supply 28 that is in electrical communication with the electrode assembly 18 through electrical couplings 30. Optionally, a controller (not shown) may be incorporated into the probe and/or with the power supply to control the delivery of energy to the heating electrodes and to provide visual and audio outputs to the physician. Some exemplary controllers are described in commonly assigned U.S. Pat. No. 6,081,749, the complete disclosure of which is incorporated herein by reference.

Exemplary embodiments of the probes of the present invention are for use in treating incontinence. Such probes will typically be substantially rigid, and sized and shaped to be insertable into a patient's vagina. In such embodiments, the distal portion will have a length between approximately 2 cm and 8 cm, and will have a width or diameter between approximately 1.0 cm and 3.0 cm. The probes can be composed of a plastic (such as polyester polycarbonate, or the like) or an inert metal (such as gold plated brass, or the like), or other bio-compatible materials that are typical of intravaginal devices. It should be appreciated however, that in alternative embodiments, the probes and guides may be partially or completely flexible. For example, in one embodiment, an electrode array may be mounted on a balloon type surface or the electrode array can be built in as features on a flexible printed circuit assembly (e.g., electrodes on flexible plastic film).

Electrodes 18 of the present invention can take a variety of forms. As illustrated in FIG. 1A, the heating electrodes can include a plurality of curved electrodes disposed on the distal portion 16 of probe body 12. In the illustrated embodiment, there are three curved electrodes 18. It should be appreciated however, that any number of electrodes and a variety of shaped electrodes can be used. A more complete description of various types of electrodes that can be used with the devices and methods of the present invention are shown and described in commonly assigned U.S. Pat. No. 6,091,995, the complete disclosure of which is incorporated herein by reference.

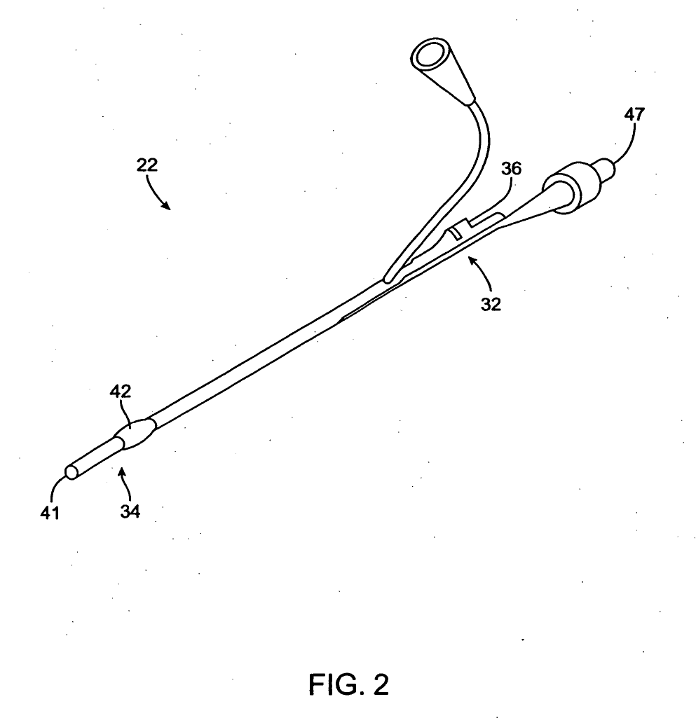

FIG. 2 illustrates an exemplary embodiment of the guide shaft 22 of the present invention that is couplable to probe body 12. Guide shaft 22 has a proximal portion 32 and a distal portion 34. In one exemplary embodiment, guide shaft 22 of the present invention is removably attached to the probe body 12 to allow for independent placement of the probe 10 and guide shaft 22 in the patient's body. A clamping structure 36, such as a series of serrations, is disposed on the proximal portion 32 to allow the guide 22 to be removably attached to the probe body 12.

While not illustrated, guide 22 can further include a temperature sensor to sense the temperature of the urethra, before, after, and during the heating treatment. Sensors may be a thermocouple, thermistor, fiber optic light based, RTD or other sensors known to those skilled in the art. The temperature sensor can be coupled to the controller to allow monitoring of the temperature of the urethral tissue. In some embodiments, if the urethra is heated beyond a predetermined threshold temperature, the controller can be configured to output a cue to the physician to inform the physician of the measured temperature. Alternatively, upon reaching a threshold temperature, the controller can be configured to stop delivery of heating energy to the electrode array.

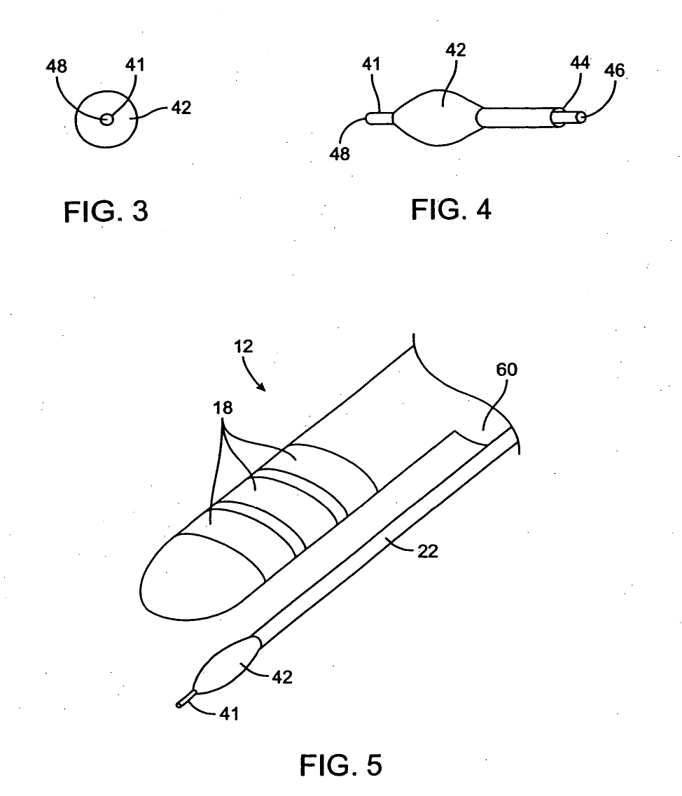

As illustrated in FIGS. 2-4, guide 22 can optionally include a tip 41 and an expansible member 42 positioned on the distal portion 34 of guide 22. Expansible member 42 can be inflated and deflated via an inflation lumen 44. Guide 22 can also include a fluid lumen 46 that has a proximal orifice 47 and distal orifice 48. In the particular configuration illustrated in FIGS. 3 and 4, the fluid lumen 46 can be coaxial with inflation lumen 44 and disposed through expansible member 42. The fluid lumen 46 can be used to deliver fluids to a body organ or to drain fluid from the body organ. Proximal orifice 47 of the fluid lumen 46 can be coupled to an aspiration or fluid source (not shown) to assist in the transfer of fluid through the fluid lumen 46. In such embodiments, expansible member 42 can be annular shaped and will have a corresponding annular inflation lumen 44 and fluid lumen 46 will be concentric or lateral with each other. It should be appreciated however, that a variety of other configurations of the lumens 44, 46 can be used without departing from the concepts of the present invention.

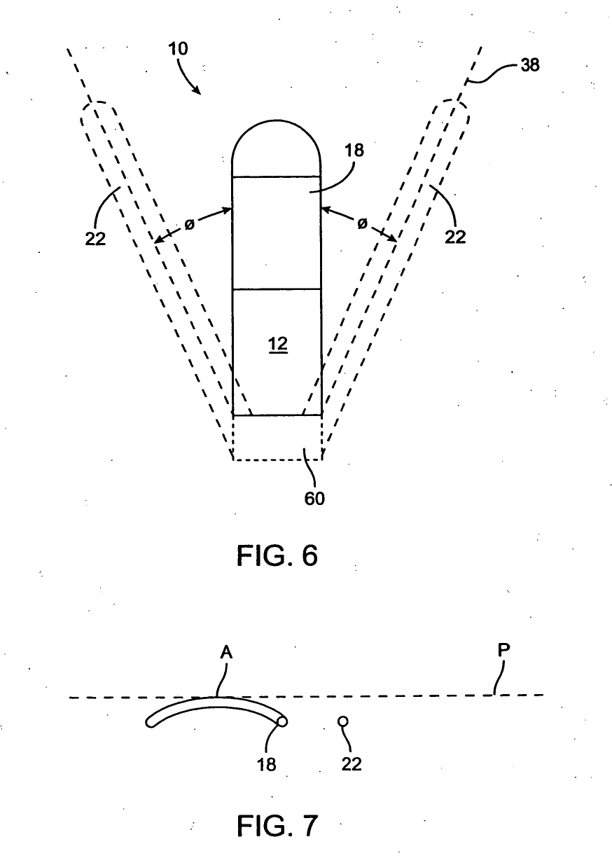

In some embodiments, urethral guide 22 can be coupled to the probe body 12 in an angled, offset configuration (FIG. 1A). Typically, a longitudinal axis 38 of urethral guide 22 will be angled from a longitudinal axis 40 of the probe body 12 (FIGS. 1A and 6). The angle θ will typically be between approximately 5° degrees and 30° degrees, and preferably approximately between approximately 11° degrees and 15° degrees. It should be appreciated, however, that in alternative embodiments, urethral guide 22 and probe body 12 may be in a parallel configuration (FIG. 5). The angled arrangement is more preferred than the parallel arrangement, because in the angled offset arrangement, as the probe is moved distally through the body orifice, the probe and guide will diverge along the angled path so that the electrodes will be positioned offset from the position of the guide and farther away from the urethra-bladder junction, which extends laterally from a longitudinal axis of the urethra.

In an embodiment most clearly illustrated in FIG. 6, a distal end of urethral guide 22 will also be positionable distal of the distal end 16 of the probe body. Thus, when the expansible member 42 of the guide extends into the bladder B, the electrodes 18 on the probe body 12 will be maintained in a position proximal of the bladder B. Such a configuration can prevent inadvertent delivery of electrical energy to the non-target bladder tissue.

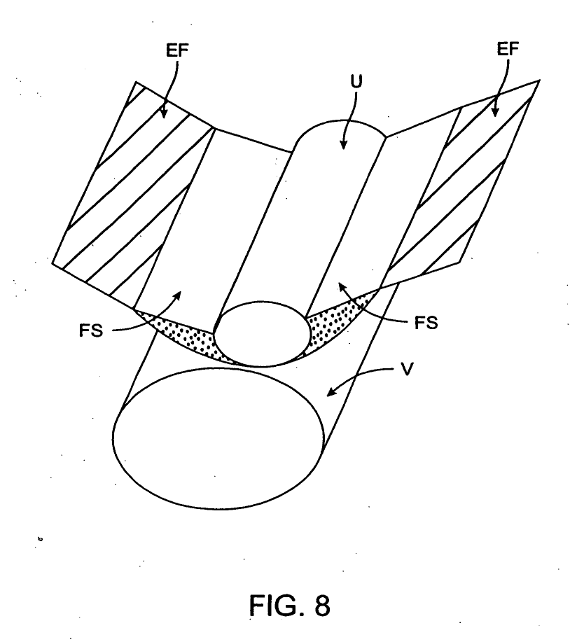

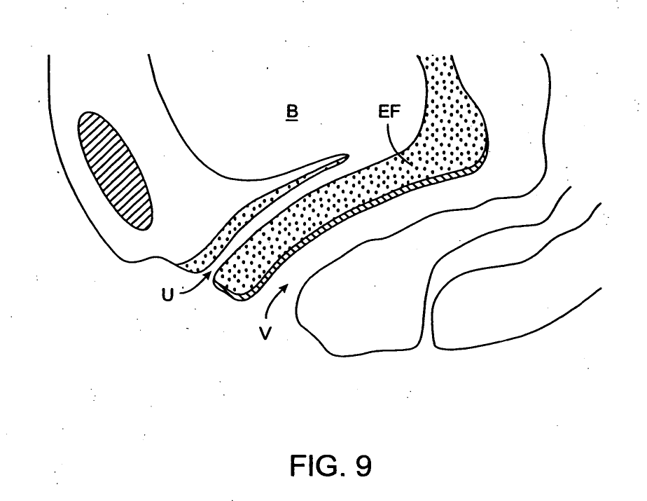

One exemplary configuration of the treatment surface 18 relative to the urethral guide 22 is illustrated schematically in FIG. 7. In such a configuration, the treatment surface 18 includes radiused electrodes that have an apex A. The guide 22 will be offset laterally from an axis of the probe body 12, typically between 5° degrees to 30° degrees, and offset below a plane P that is orthogonal/tangent to the apex A (or parallel to an upper plane of a planar electrode). By offsetting the distal end of the guide 22 below the top plane of the electrode, the guide 22 can tension the vaginal surface tissue engaged by the probe body 12 and bias the electrodes 18 into contact with the target tissue. Such a biasing configuration can improve the delivery of the electrical energy from the electrodes 18 into the target tissue and reduce the chance of delivering energy to non-target tissue.

In one embodiment, guide 22 can be rigidly coupled to probe body 12 with a coupling assembly 60 so as to maintain a rigid assembly. By maintaining a substantially rigid connection, rigid guide 22 can properly position electrodes 18 offset laterally from a sensitive non-target tissue, such as the urethra, so that delivery of electrical energy through the electrodes 18 is sufficiently spaced from the non-target tissue.

In some configurations, the coupling assembly 60 of the present invention can be configured to allow attachment to the probe body along both sides of the probe body. As shown in FIG. 6, urethral guide 22 can be positioned laterally along either the left or right side so as to allow contact of the electrodes 18 with tissue laterally to the left or right of the urethra.

The coupling assembly 60 of the present invention can provide an attachment between the guide 22 and the probe body 12 that allows the user to attach and detach the guide to position the electrodes adjacent the target tissue. One exemplary coupling assembly is illustrated in FIG. 1B. The coupling assembly includes a substantially symmetrical left and right pockets 62, 64 that can receive a proximal end of the urethral guide 22. A rotatable guide clip 66 having a left and right coupling handles 68, 70 is disposed between left pocket 62 and right pocket 64. The left pocket 62 and right pocket 64 can include a serrated mount 72 that can interact with clamping structure 36 on the proximal end of the guide 22. Additionally, the pockets 62, 64 can include a snap feature 74 that can interact with the left and right coupling handles 68, 70 to lock the guide 22 within the pockets.

The urethral guide can enter the pockets either by vertically or axially sliding the proximal end of the urethral guide 22 into a selected pocket. In exemplary embodiments, the proximal end of the urethral guide 22 includes matching serrations (not shown) that match the serrated mount 72 in the pocket so as to allow for incremental axial positioning of the urethral guide with respect to the applicator and handle. After the guide 22 is positioned in a desired axial position, the selected handle 68, 70 can be secured by snapping it into the snap feature 74.

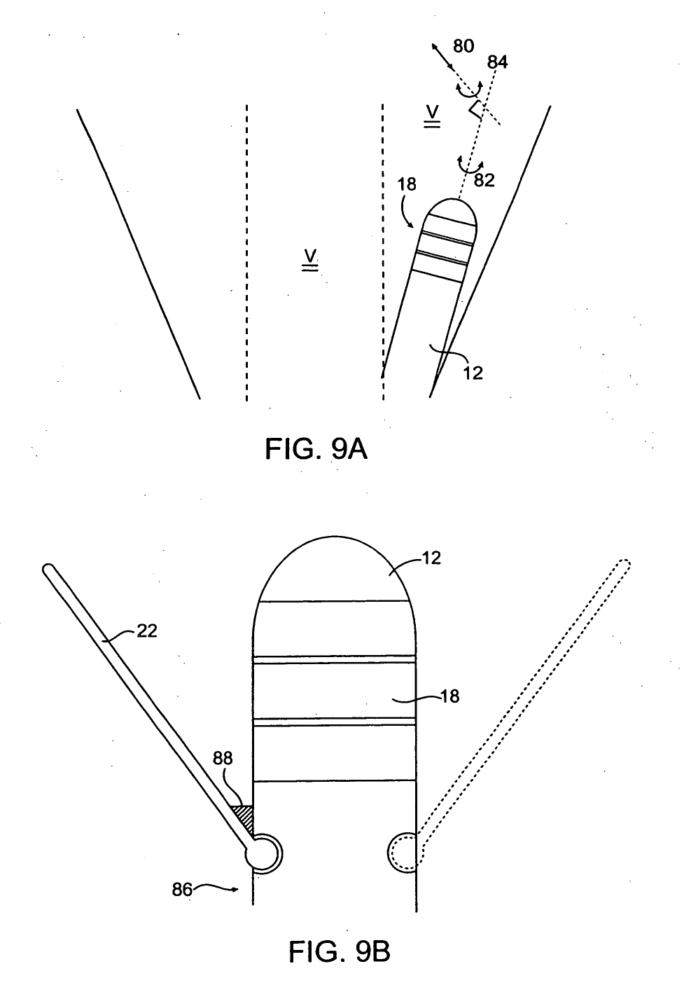

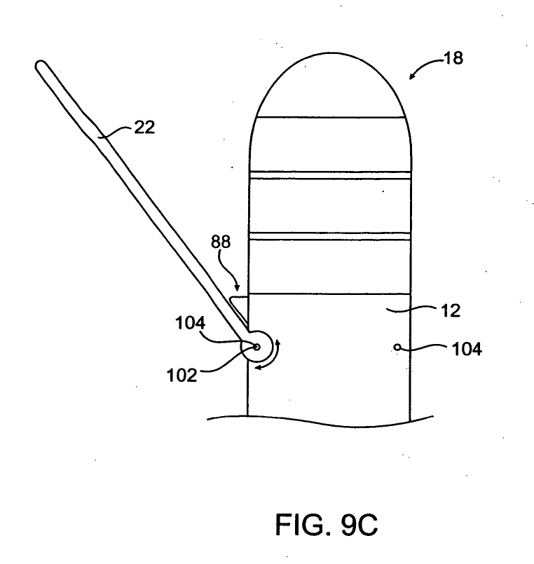

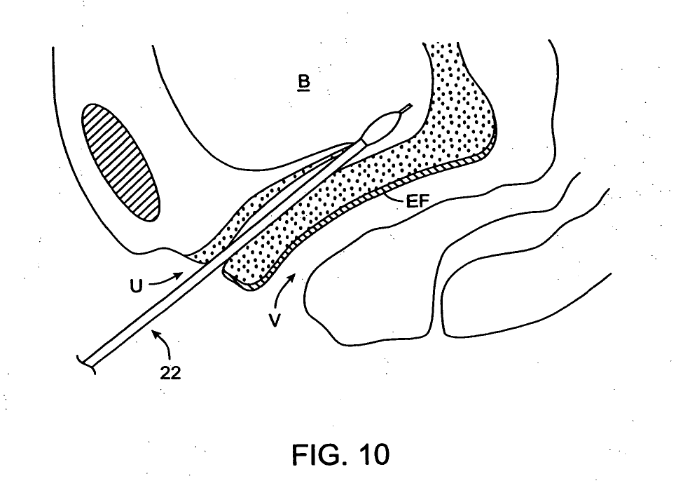

FIGS. 9A to 9C illustrate an embodiment of the probe and urethral guide 22 that allows the operating physician the flexibility of changing the position of the urethral guide 22 relative to the probe body 12. As illustrated in the top view FIG. 9A, it is preferred to position the treatment surface 18 of the applicator in a laterally offset position relative to the urethral tissue U. In one embodiment, the urethral guide can be coupled to probe body 12 in a manner that allows the physician to place the treatment surface in different orientations lateral to the urethra tissue U. As illustrated by the arrows in FIG. 9A, in some embodiments, the treatment surface 18 will be rotatable about one or more axes and/or movable in at least one direction. For example, in one embodiment, the urethral guide can be movable in at least one of an up/down direction 80, rotation about a longitudinal axis of the probe body 82, and rotation about an axis perpendicular to the longitudinal axis 84 (e.g., pivot around a distal portion of the probe body).

In the embodiment illustrated in FIG. 9B, probe body can be coupled to the urethral guide 22 with a ball joint 86 or other joint that allows rotation of the guide about at least some of the degrees of freedom 80, 82, 84. In some configurations, probe body 12 can include a physical stop 88 that limits the pivoting of the urethral guide 22 to prevent the urethral guide from being positioned below a minimum angular offset, (e.g., 11 degrees). Preventing the urethral guide from going below a minimum angular offset can prevent the treatment surface from being aligned with the urethral tissue U and fascia sheets. As illustrated further in FIG. 9B, ball joint 86 can be disposed on the left and/or right side of the probe body 12 so as to allow treatment on the tissue that is laterally to the left and right of the urethral tissue.

The ball joint 86 can be implemented in a variety of ways. For example a proximal end of urethral guide 22 can include a ball, while probe body 12 can include a socket with a cover so as to removably capture and rotatably hold the ball within the socket. In another example the proximal end of urethral guide 22 can include pins or other protrusions that can be retained in a dimple that is in the joint of the probe body 12 so as to rotatably couple the urethral guide to the probe body.

If it is desirable to only pivot the urethral guide 22 about one axis, a simple joint 98 can be used to couple the urethral guide 22 to the probe body 12 so as to allow rotation 100 about a single axis. As can be appreciated, there are a variety of conventional methods of rotatably attaching the urethral guide 22 to the probe body 12. In the illustrated example in FIG. 9C, urethral guide 22 includes a hole 102 that can mate with a pin 104 on the probe body 12. In such embodiments, the urethral guide can be removable or non-removable and the urethral guide 22 can be attached to the left and/or right side of the probe body 12.

It should be appreciated however, that other conventional attachment means can be used to attach the urethral guide 22 to the probe body 12. For example, the guide 22 and probe body 12 can be coupled with a threaded attachment, a toggle clamp mechanism for pressing a clamping surface of the guide against the probe body, a sliding latch mechanism clip, a ¼ turn fastener, or the like.

In some embodiments of the methods of the present invention, probe body 12 will be configured to be insertable in a second body orifice, while guide shaft 22 will be configured to be inserted into a first body orifice so as to accurately position the probe body 12 and electrodes 18 adjacent a target tissue in the second body orifice. Preferably, the probe body 12 will be positioned in an offset position relative to the guide 22. In a particular method, the guide shaft 22 is configured for insertion into a patient's urethra U while the probe body 12 will be configured for insertion into a patient's vagina V (FIGS. 8 and 9). In such embodiments, urethral guide 22 will generally have a diameter and length that allows a distal end 34 of the urethral guide 22 to extend through the patient's urethra U and into the patient's bladder B. As such, the urethral guide will have a length between approximately 3 inches and 6 inches and a diameter between approximately 0.12 inches and 0.38 inches.

As illustrated in FIGS. 8 and 9, the urethra U is supported by triangular shaped fascia sheets FS that have nerve bundles. Delivery of electrical energy into the fascia sheets FS is undesirable. The electrical energy is preferably delivered to the endopelvic fascia EF that is spaced laterally to both sides of the urethra. To offset the probe 12 away from the fascia sheets and urethra, a longitudinal axis of guide 22 can be aligned in an angled arrangement with a longitudinal axis of the probe body 12. The angled offset moves the probe body laterally (left or right) away from the urethral tissue and fascia sheets and adjacent the target endopelvic fascia EF for treatment. Because of the offset configuration between guide 22 and probe 12, the electrodes 18 will be offset from urethra U and moved against the target tissue that is laterally spaced from the urethra (FIG. 8). In order to provide accurate positioning, in some embodiments, urethral guide 22 is substantially rigid so as to maintain its relative position between the electrode 18 and guide shaft 22. As such, guide 22 is also typically in the form of a rigid shaft. In some embodiments, rigid guide 22 is at least partially composed of or covered with a bio-compatible material that is typical of intraurethral catheter devices. If the guide shaft is too flexible, then the position of the electrodes 18 relative to the guide shaft 22 may not be maintained in the desired position and electrical energy may be inadvertently delivered to non-targeted tissue (e.g. urethra and nerve bundles surrounding urethra).

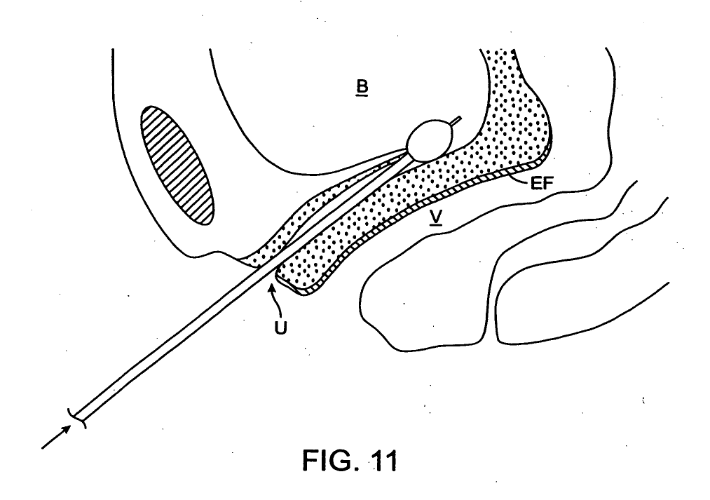

An exemplary embodiment of a method of the present invention is illustrated in FIGS. 10-13. In a noninvasive medical procedure to treat incontinence, the urethral guide 22 can be inserted into the urethra U (FIG. 10). During its distal movement through the urethra U, expansible member 42 will be in its deflated configuration. Once the expansible member enters the orifice to the bladder B, expansible member 42 can be inflated to “lock” the position of the urethral guide 22 to prevent proximal retraction of the urethral guide 22 out of the bladder B (FIG. 11). In some embodiments, the urethral guide can include markings to ensure that the urethral guide remains in the most proximal position allowed by the expansible member relative to the bladder neck orifice. If desired, any liquid that is present in the bladder B can be drained out of the bladder B through the distal orifice 48 and fluid channel 46 within the urethral guide.

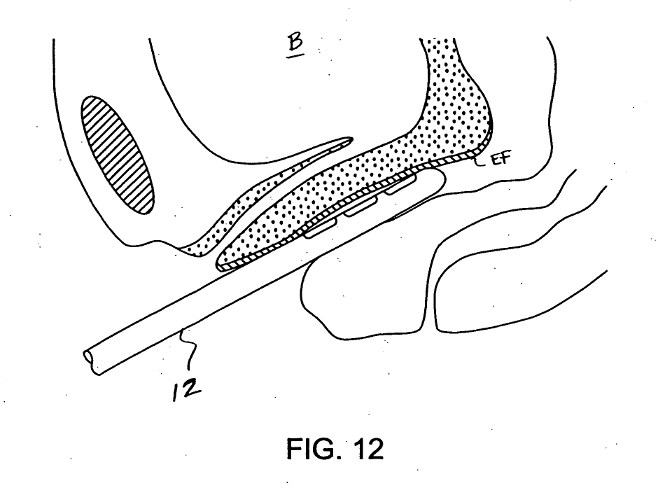

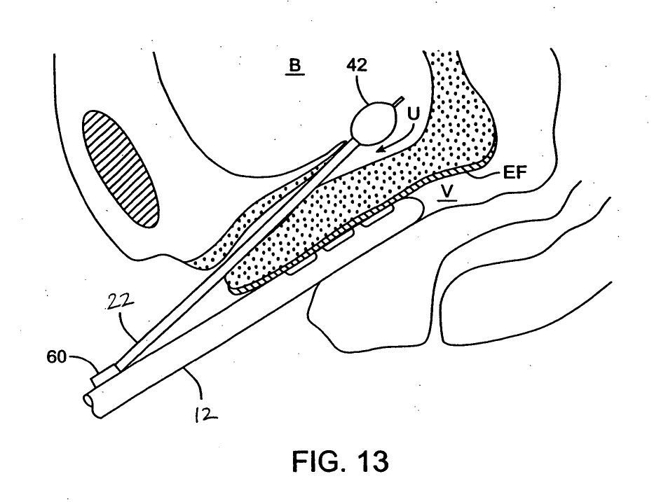

FIG. 12 illustrates that the probe body 12 can be inserted into the patient's vagina V (for clarity guide 22 is not shown). Once it is grossly determined that the probe has been inserted to the proper location the urethral guide and probe body can be attached together with the coupling structure 60 (FIG. 13). Such coupling will ensure that the distal tip of the probe body 12 is maintained proximal of the distal end of the guide 22 so as to position the treatment surface adjacent the target endopelvic fascia EF and to prevent the electrodes from delivering electrical energy to the bladder or other non-target tissue. The coupling structure also will maintain the offset configuration between the axes of the guide 22 and probe body 12 so as to position the electrodes offset laterally away from the urethra and towards the target tissue EF. Optionally, if the guide 22 is positioned below a top plane of the electrode, the guide may tension the tissue and bias the electrodes 18 into the target tissue EF.

While FIGS. 10 and 12 illustrate the urethral guide 22 and probe body 12 being separately inserted into the body orifices, it should be appreciated that in alternative embodiments, the urethral guide 22 and probe body 12 can be simultaneously inserted into the urethra U and vagina V while fixedly or rotatably connected with coupling structure 60, 86.

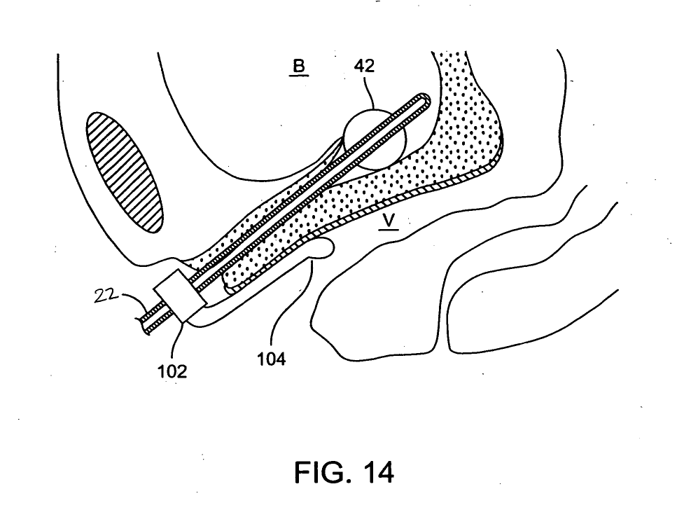

Some alternative methods of registering the urethral guide and probe will now be described. FIGS. 14 to 18B illustrate other embodiments of probe 12 and urethral guide 22 of the present invention that incorporate a passive registration assembly to position probe 12 in a position relative to urethral guide 22 so as to position the treatment surface 18 adjacent the target tissue. In the illustrated embodiments, urethral guide 22 is configured to be maintained in a detached position relative to probe 12. Urethral guide 22 and probe 12 can include landmarks such as an expansion member, palpation member, or other sensors or transmitter markers that indicate a mid urethra point. The marker(s) can be placed in the vagina or the marker can be placed in the urethra and sensed through the vaginal wall.

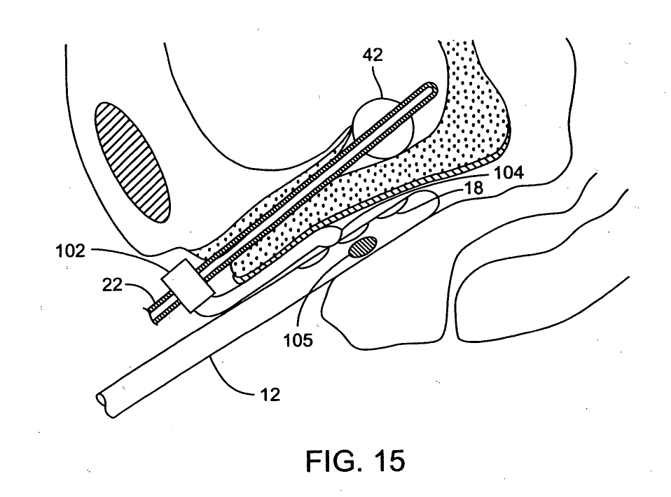

In the embodiment illustrated in FIGS. 14 and 15, a physical marker can be used to help position probe 12 relative to urethral guide 22. While probe 12 and urethral guide 22 are not physically connected, the relative position and/or spacing of the probe 12 and urethral guide 22 can be used to indicate to the physician as to whether or not the treatment surface 18 of probe 12 is positioned adjacent the target tissue.

After urethral guide 22 is positioned in the urethra U, a bobby-pin type clip or a U-clip 102 can be coupled to the urethra guide to provide a physical marker in the vagina for the physician. In one embodiment, U-clip 102 can include a palpation member 104 at a distal end that will be positioned in the vagina to allow the physician to feel the mid-urethra point. In such embodiments, probe 12 can also include a corresponding palpation members 105, such that when the probe is inserted into the vagina, the physician can proximally/distally align and laterally offset palpation markers 104, 105 so as to position the treatment surface adjacent the target tissue and offset from the non-target urethral tissue.

Palpation members 105 can be opposed bumps or indentations, an enlarged portion of probe body, an embossed marking, or any other element that allows the physician to determine by physical contact, a position of the treatment surface 18. In one embodiment, palpation members 105 will be on opposite sides of the probe body and separate from the treatment surface 18. In other embodiments, however, the palpation members 105 can be positioned on other surfaces of the probe body, such as on the treatment surface 18 or the like.

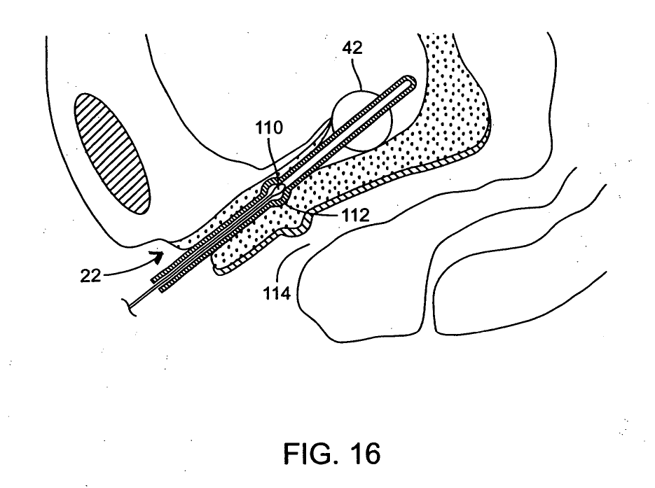

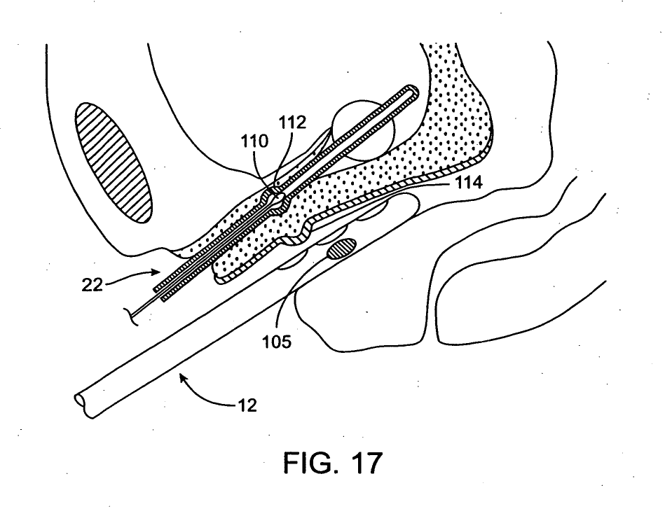

In the embodiments illustrated in FIGS. 16-18B, instead of providing a marker in the vagina, the urethral guide 22 can be configured to provide a marker of the mid-urethra point through the vaginal wall. For example, as shown in FIG. 16, urethral guide 22 can include an expansion member 110 that creates an expanded region 112 in urethral guide 22. Expanded region 112 will be sized so as to create a discernible bulge or bump 114 in a vaginal wall. The physician can then manually feel along the upper vaginal wall to find bulge 114 and use bulge 114 as a marker for the palpation members 105 on probe 12. Similar to above, as shown in FIG. 17, the physician can then position the treatment surface in a laterally offset and proximally/distally aligned position relative to bulge 114 by aligning palpation members 105 with bulge 114 and positioning the treatment surface adjacent the target tissue in the vagina.

In one embodiment, palpation members 105 can be positioned laterally from the bump 114 or palpation member 104 between approximately 1 cm and 2 cm and should not be positioned proximal or distal of the bump. As can be appreciated, however, it may not always be possible to proximally/distally align the palpation members 120 with bump 104, and a proximal or distal offset of between approximately±5 mm may be acceptable for delivering a treatment to the target tissue.

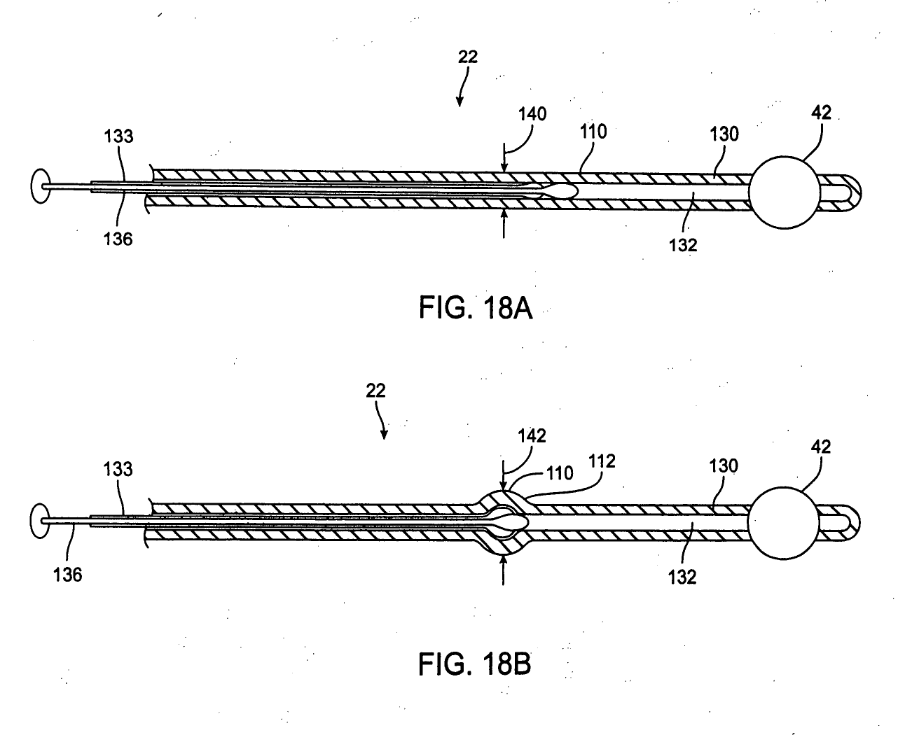

FIG. 18A illustrate one embodiment of a simplified urethral guide in a relaxed position and FIG. 18B illustrates the urethral guide in an expanded position. Urethral guide 22 includes an expansion member 110 and an outer tubular member 130 that defines at least one inner lumen 132. A second tubular member 133 can be disposed within lumen 132 such that an expandable region 112 will be positioned near a center point of urethral guide 22. Positioning can be achieved by first measuring the urethral length with a marked urethral guide and pullback of the distal balloon 42 to the bladder neck. Marks on the inner lumen of the urethral guide permit its insertion to the correct distance based on the then known patients urethral length. An elongate shaft 136 can include the expansion member 110, such as a wedge, balloon, or the like, at or near its distal end. Elongate shaft 136 can be movably disposed within lumen 132 such that proximal actuation of elongate shaft 136 by the physician moves expansion member 110 into expandable region 112 so as to enlarge the diameter of outer tubular member 130 from a first width 140, to a second, larger width 142 (FIG. 18B). The expansion of the outer tubular member 130 can be used to create bulge 114 in the vaginal wall.

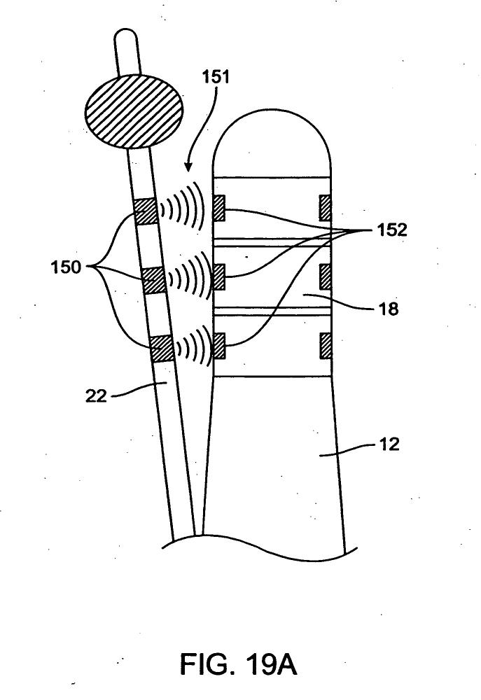

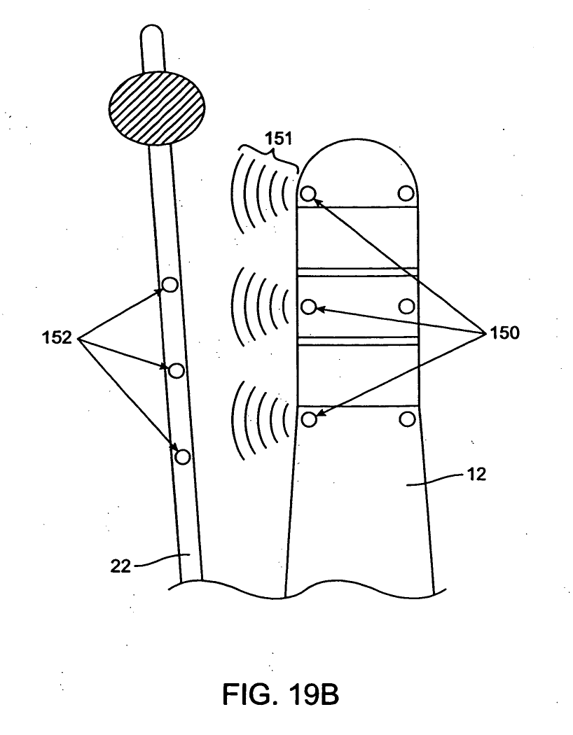

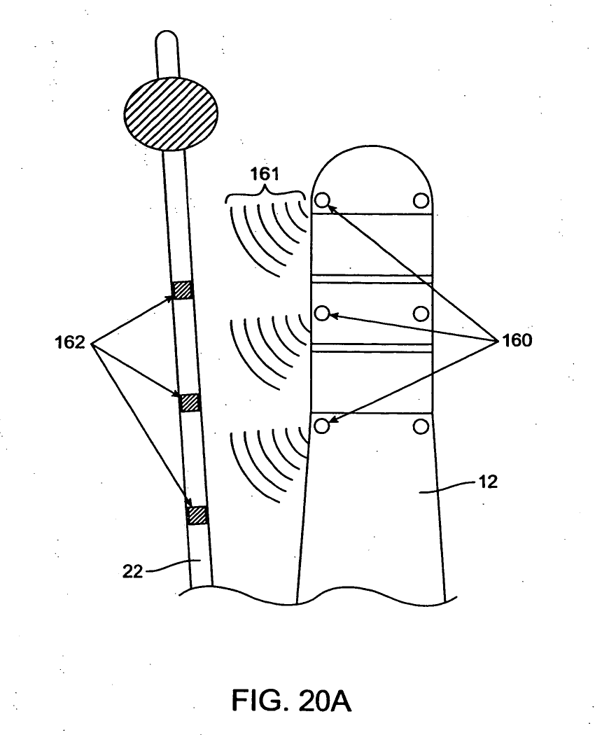

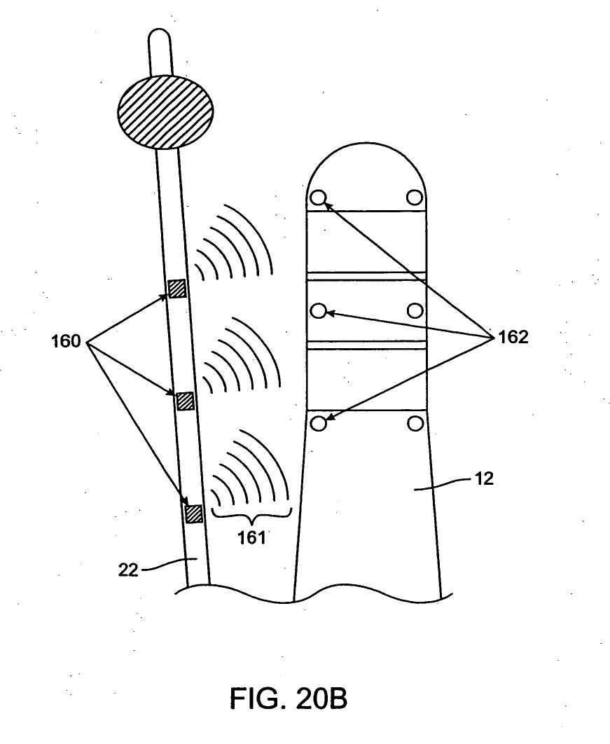

FIGS. 19A to 20B illustrate other embodiments of urethral guide 22 and probe body 12 which utilize an automatic electromagnetic coupling to assist the physician in positioning the probe body 12 adjacent the target tissue. In the embodiment illustrated in FIG. 19A, an RF coupling can be used to transmit and receive RF energy waves 151 to monitor the position of the probe relative to the urethral guide. One or more RF transmitters 150 can be coupled to urethral guide 22 to generate RF energy waves 151. In the illustrated embodiment, a plurality of RF transmitters 150 are positioned around a portion of guide 22 that will be positioned at the mid-urethra. Probe body 12 can include one ore more RF receivers 152. In the illustrated embodiment, probe body 12 can include a plurality of RF receivers that are positioned around the treatment surface. While the RF receivers 152 are illustrated on the treatment surface, it can be appreciated that the RF receivers 152 can be positioned within probe body 12, along a bottom surface of probe body, and/or separate from RF receivers. RF receivers 152 need only be positioned on probe body 12 to indicate the relative position of the treatment surface.

In another embodiment, illustrated in FIG. 19B, the RF transmitters 150 can be positioned on probe body 12 while RF receivers 152 can be positioned on urethral guide 22.

FIGS. 20A and 20B illustrate another embodiment of probe 12 and guide 22 which use an magnetic coupling to register the probe body 12 with guide 22. Similar to above, the embodiment illustrated in FIG. 20A, the urethral guide 22 can include one or more magnetic source(s) 160, such as a magnet to generate a magnetic field 161. Probe body 12 can include one or more magnetic field sensors 162, such as a Hall Effect Sensor to sense the strength of the magnetic field 161 created by the magnetic sources 160. The strength of the magnetic field generated by magnetic source 160 and sensed by the magnetic sensors 162 will produce a signal that is proportional to the spacing between the source 160 and sensors 162. The magnetic field can be sensed by sensors 162 and the signal from the sensors can be transmitted to a controller CPU (not shown) to determine the position of the probe 12 relative to the urethral guide 22.

As illustrated in FIG. 20B, in an alternative embodiment, the magnetic sensors 162 can be positioned on urethral guide 22 and magnetic sources 160 can be positioned on probe body 12.

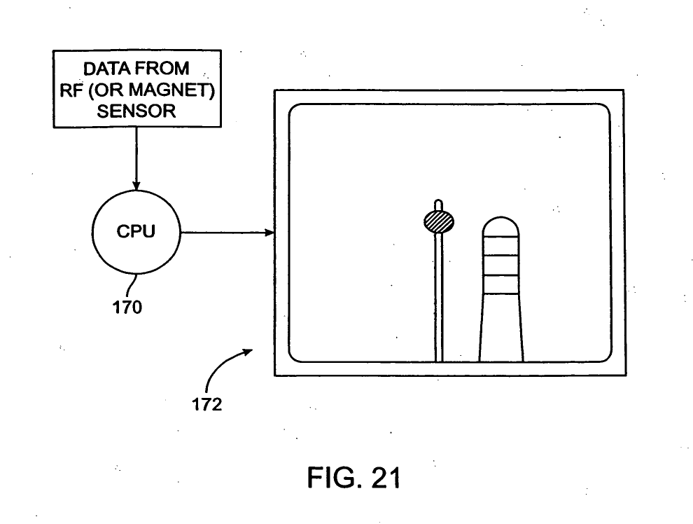

In any of the electromagnetic coupling embodiments, the transmitters 150, 160 will emit an position signal that will be received by sensors 152, 162 that will indicate the relative position of the probe body 12 relative to urethral guide 22. As illustrated in FIG. 21, in some embodiments, the data from the sensors can be transmitted to a CPU 170 of controller so as to generate a graphic representation of urethral guide and probe body on an output display 172. CPU 170 can analyze the real-time data received from the sensors to provide direct feedback to the physician regarding the probe body 12 location within the patient's vagina.

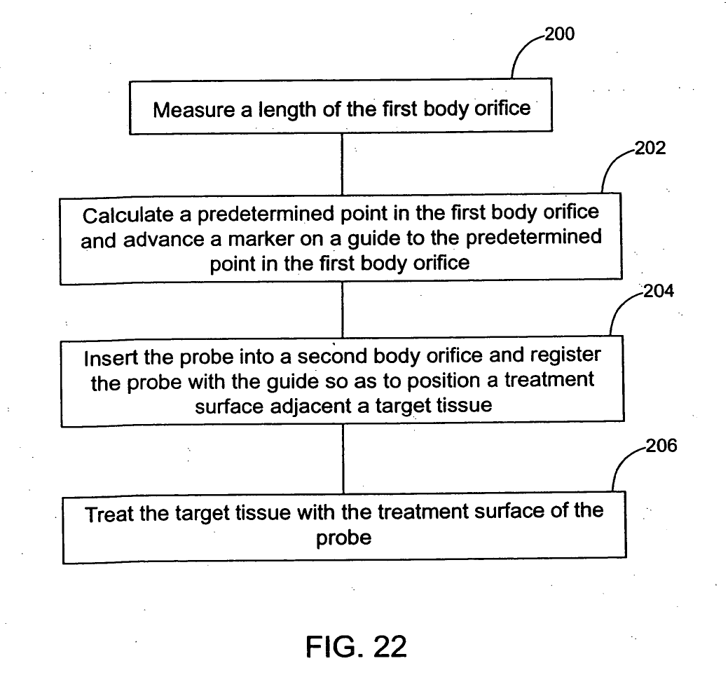

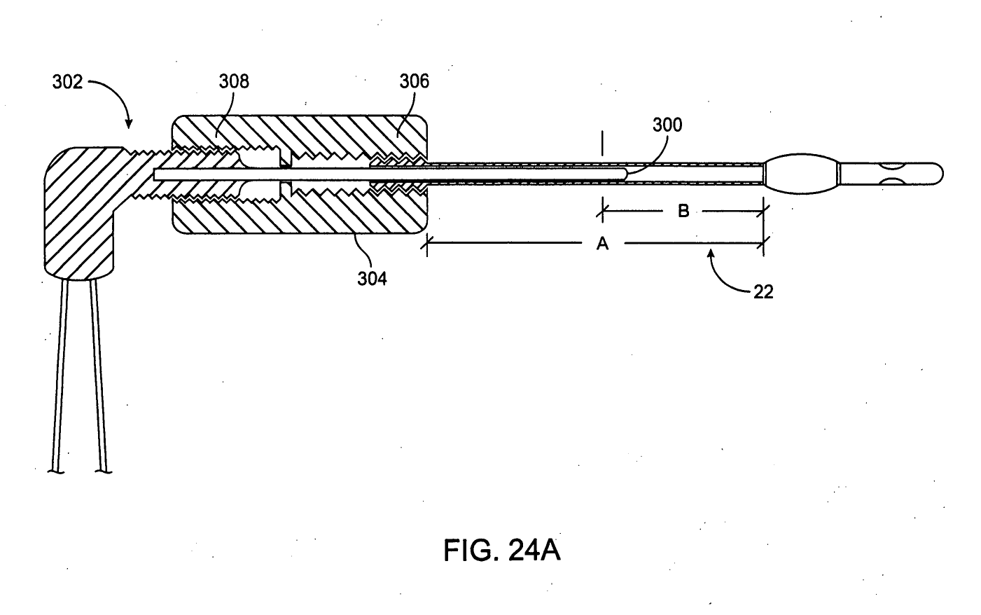

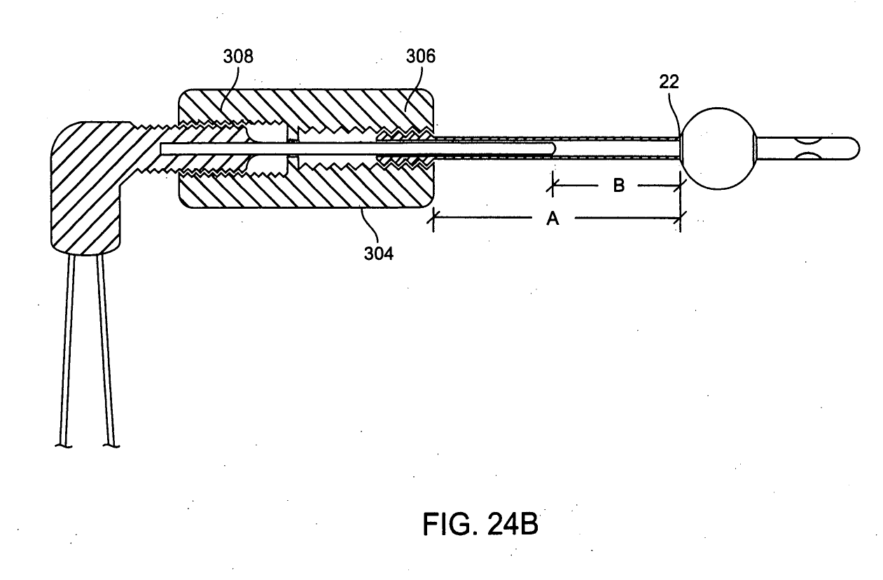

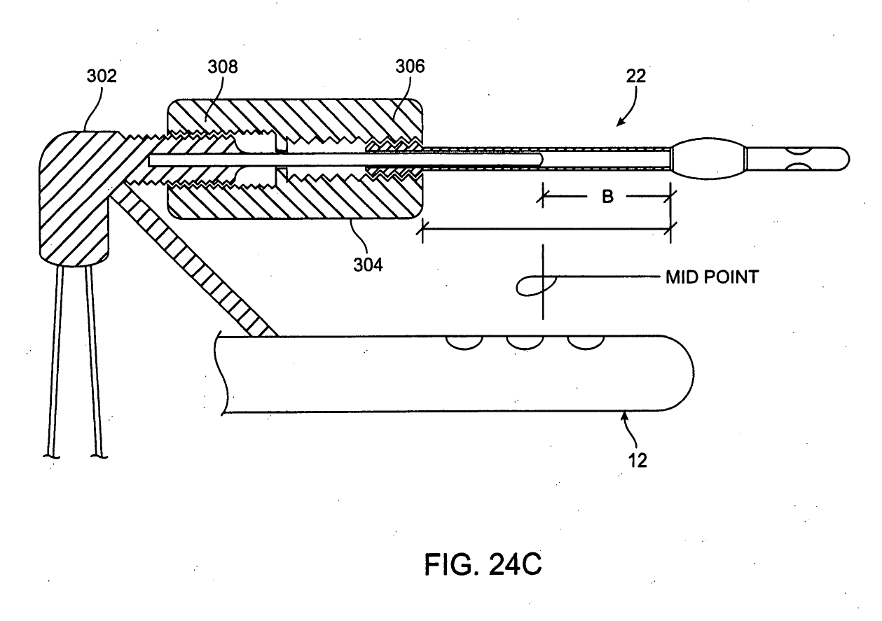

Some embodiments of the methods of the present invention will now be described. As illustrated schematically in FIG. 22, some methods of the present invention include the step of measuring a length of the first body orifice (e.g., urethra), 200. In some embodiments such as that shown in FIGS. 24A to 24F, it may be possible to directly place the sensor or palpation device at the mid-urethra position without measuring the length of the first body orifice.

After the length of the first body orifice is determined, a marker (e.g., transmitter, receiver, or physical marker) of the guide can be advanced into the first body orifice and positioned at a predetermined point (e.g., halfway into the length of the urethra or the mid-urethra) which will allow for proper positioning of the probe, 202. After the guide has been properly positioned, the probe can be inserted into the second orifice and registered with the guide, 204. After the probe has been placed in a predetermined position relative to the guide, the target tissue can be treated with a treatment surface of the probe, 206.

A variety of conventional and proprietary methods can be used to measure the length of the first body orifice and to calculate the predetermined distance. For example, in the embodiments in which the first body orifice is the urethra, the physician may manually measure the length of the urethra and then calculate the mid-urethra point (approximately half the length of the urethra).

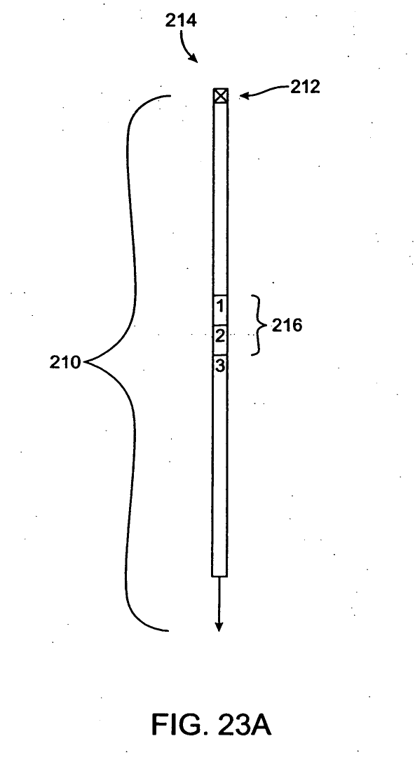

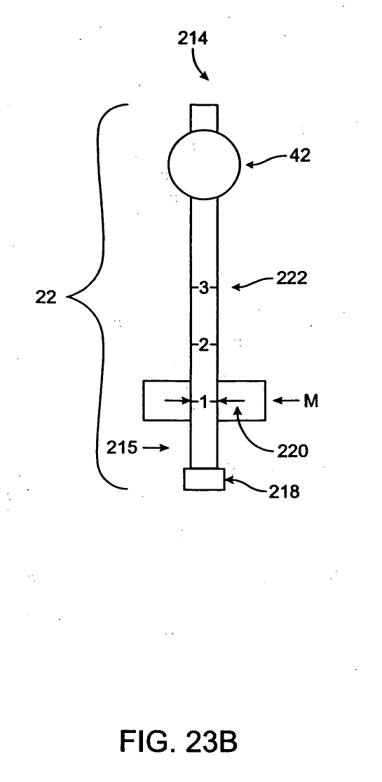

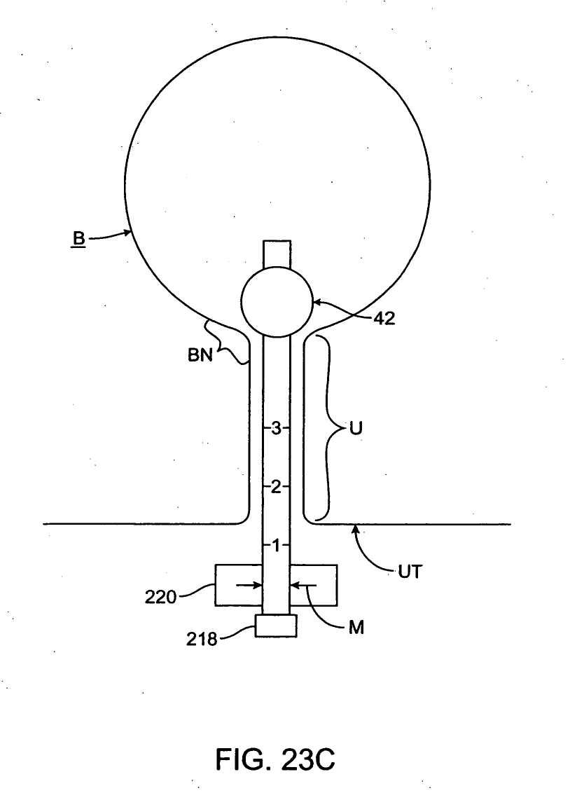

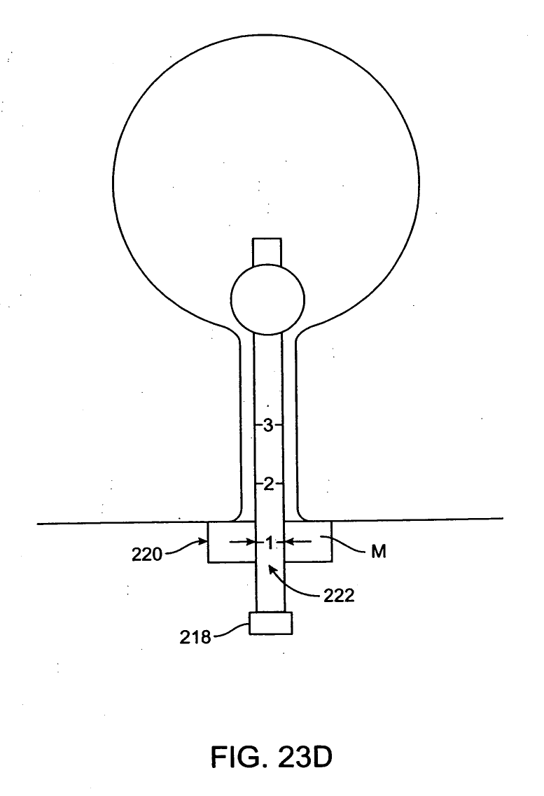

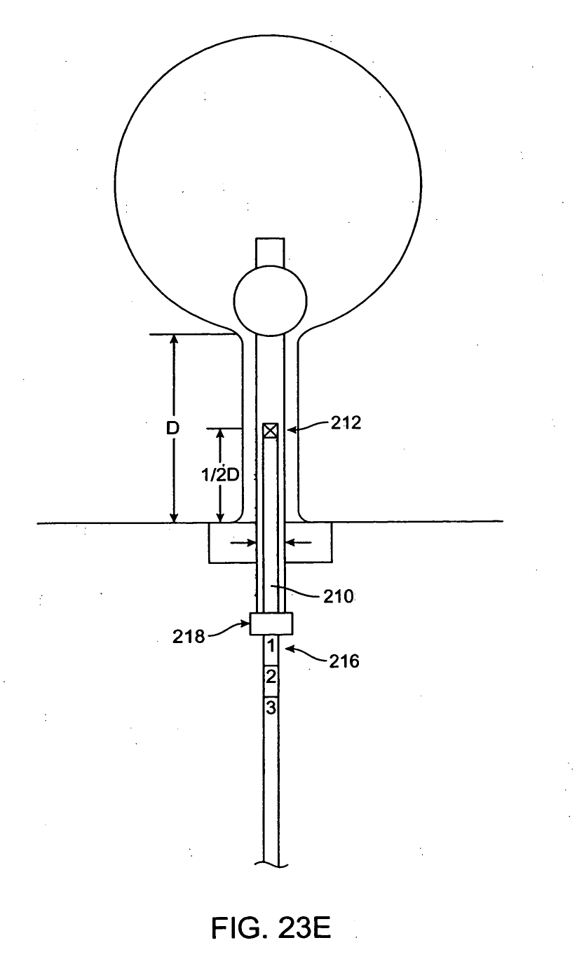

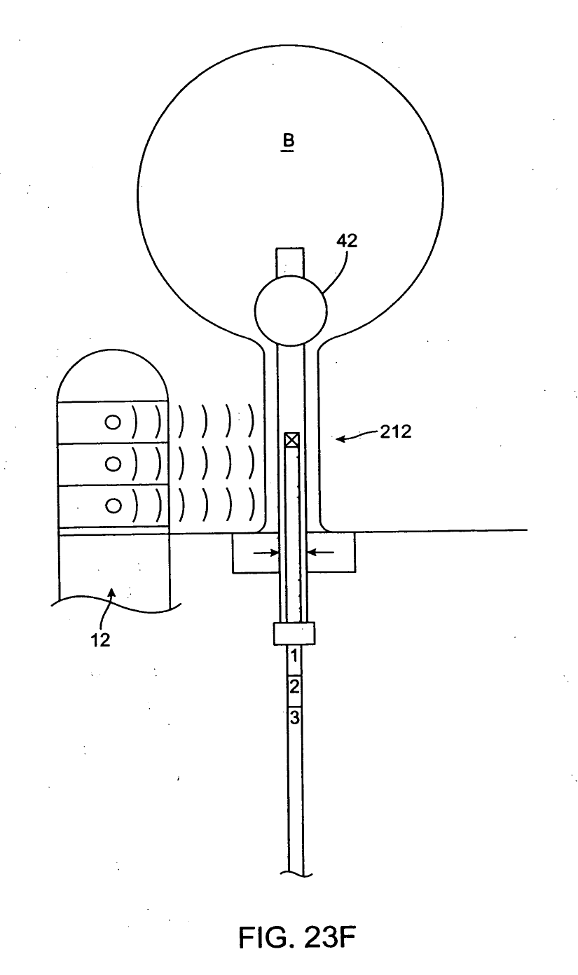

One embodiment of a device and method for measuring the length of the urethra and locating its midpoint is illustrated in FIGS. 23A to 23F. The device comprises a sensor rod 210 that includes one or more sensors 212 at or near its distal end 214. Sensor rod 210 can fit within an inner lumen of guide shaft 22. Sensor wires can run through a lumen of the sensor rod to communicate with the controller. Sensor rod 210 can include positioning graduations 216 that assist the physician in positioning the sensor(s) at the mid-urethra.

As shown in FIG. 23B, urethral guide 22 can include a balloon 42, a locking mechanism 218 around its proximal end 215 and a sliding stop 220 that can fit over urethral guide 22. Sliding stop 220 can include a marker M, such as an arrow that is configured to align with graduations 222 on the outer surface of the urethral guide to indicate the urethral length.

After the urethral guide is inserted into the urethra U and locked into the bladder B with balloon 42, the urethral guide can be pulled proximally to seat balloon 42 against the bladder neck BN. Thereafter, the sliding stop 220 can be pushed distally until it contacts the outer surface of the urethra tissue UT or urethra meatus (FIG. 23C). As shown in FIG. 23D, once the sliding stop has reached the urethral tissue, the sliding stop can be locked into place using spring force on a squeeze clip, expansion pins or a thumbscrew or other similar mechanisms known to those skilled in the art and the graduation 222 that is aligned with marker M can be read.

As shown in FIG. 23E, the sensor rod 210 can then be inserted into the inner lumen of the urethral shaft until the graduation 216 that matches the graduation 222 on the guide that is aligned with marker M is aligned with locking mechanism 218. In such a position, sensors 212 will be positioned at approximately the midpoint of the measured length of the urethra. The sensor 212 (or transmitter) can be used to measure or generate a position signal to indicate the position of the mid urethra, as described above (FIG. 23F).

In another embodiment, the methods and device illustrated in FIGS. 24A to 24C can be used to automatically place a sensor or palpation device at the mid urethra position once the device is adjusted to equal the total length A of the patient's urethra. As shown in FIG. 24A, urethral guide 22 can include a movable marker 300 such as an RF/magnetic transmitter or receiver, or an expansion member disposed within a lumen of urethral guide 22 that is coupled to a rotating adjustment assembly 304. A stationary proximal body 302 can be coupled to the urethral guide 22 via the rotating adjustment assembly 304. In the illustrated embodiment, the position of the marker 300 can move as the adjustment assembly is rotated and moved axially and will always be positioned at a half-way point B of the distance A.

In the illustrated embodiment, a proximal end of urethral guide 22 can include a 2X-pitch screw thread 306 and a distal end of proximal body 302 can include fine pitch screws that have an X-fine pitch screw threads 308. Thus, in the illustrated embodiments in FIGS. 24B and 24C, the urethral guide 22 can be inserted into the urethra and the adjustment assembly 304 is rotated and moved into contact against the urethra meatus, such that the length between the balloon and the distal end of the adjustment assembly will be equal to A which is then equal to the patients urethral length. The marker 300 can maintain its center position at the mid-urethra point B due to the 2:1 pitch difference of the threads 306, 308 and the sensor or transmitter on the probe body 12 can be positioned adjacent the mid-urethra point, as described above. Thereafter, the probe body 12 can be inserted into the patient's vagina and positioned adjacent the target tissue, using any of the above recited methods.

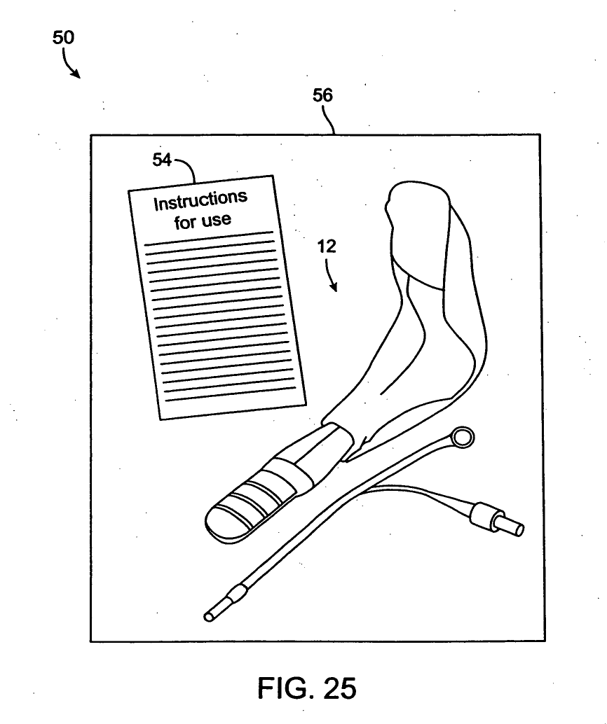

Referring now to FIG. 25, a kit 50 includes a probe 12, a guide 22 and instructions for use 54. Probe 12, guide 22, and instructions 54 can be placed in packaging 56. Guide 22 can be any of the embodiments described above, and instructions 54 can set forth the steps of one or more of the methods described herein for heating and shrinking or stiffening tissue for treating urinary incontinence. Additional elements of the above described systems may also be included in packaging 56, or may alternatively be packaged separately.

Instructions 54 will often comprise printed material, and may also be found in whole or in part on packaging 56. Alternatively, instructions may be in the form of a recording disk, CD-ROM or other computer-readable medium, video tape, sound recording, or the like.

Referring now to FIGS. 26A through 26D, an exemplary embodiment of the urethral measuring positioning guide 22 is shown. FIGS. 27A and 27B illustrate an exemplary embodiment of the treatment probe 12. This exemplary palpation system 12, 22 for urinary incontinence treatment helps facilitate registration of the treatment probe 12 along a urethral axis so as to position a treatment zone 400 of the probe 12 adjacent a target support tissue of a patient. In particular, this palpation system 12, 22 avoids inadvertent damage to nerves and/or other tissues by safely separating the treatment probe 12 away from the nerves and/or tissues in the area of the bladder neck, bladder, urethral meatus, vaginal meatus, urethra, and other incontinence-effecting nerves and/or tissues. Ideally, the physician will also have some freedom to move the treatment probe 12 manually as desired to achieve the best thermal contact, electrical contact, ergonomic fit to the patient, or the like, while maintaining an acceptable registration region.

As shown in FIGS. 26A through 26D, the urethral guide 22 comprises a proximal portion 404, a distal portion 402, and an axial inflation lumen 406 therebetween. The guide 22 includes a first palpation member 408 that is positioned on an outer surface between the distal and proximal portions 402, 404 and in a fixed relationship to an anatomical landmark, such as a bladder neck. This fixed relationship may comprise a distance in a range from about 12 mm to about 20 mm. It will be appreciated that this distance is dependent on a variety of factors, including the patient's urethral length, a length of the probe treatment zone 400, and the probe 12 geometry. For example, in one embodiment the fixed distance between the first palpation member 408 and the bladder neck is about 15 mm, which is appropriate for urethral lengths of about 32 mm and higher. The guide 22 further includes an expandable body such as an elastomeric balloon 42, which has been described above in detail, on a distal portion thereof 402. After transurethral insertion, the expandable body 42 may be expandable within the bladder via the inflation lumen 406 which further includes a proximal orifice 412 and an internal valve 414 disposed thereon. Once expanded, the balloon 42 is seated against the bladder neck. This allows for the fixed positioning of the first palpation member 408 relative to the bladder neck and the balloon 42.

The guide 22 further includes a plurality of graduations or markers 410 on an outer lumen surface near the proximal portion 404. These markers 410 allow a physician to measure and confirm the patient urethral length prior to the treatment procedure. Urethral measurement ensures that the selected treatment probe 12 is appropriate for the patient's urethral length so as avoid inadvertent treatment outside of the registration region, for example the bladder, bladder neck, urethral meatus, or vaginal meatus. The guide 22 further includes a meatus engaging surface or retention stop 416 on the proximal portion 404 and movably coupleable to the inflation lumen 406. This stop 416 is oriented distally for engaging a urethral meatus via the adjustment knob 418 which is rotatable so that the adjustable screw length markers 420 correspond to the measured urethral length. An attachment clip 422 is further provided on the stop 416 which is connectable to a retention strap attached to the patient. The inflated balloon 42, retention stop 416, and retention strap ensure that the guide 22 is maintained in a stable horizontal position.

FIG. 26A shows a side view of the urethral guide 22 described above, wherein the balloon 42 is shown inflated and the meatal stop 416 is at the most distal position at a urethral length of 33 mm. FIG. 26B illustrates an isometric view of the urethral guide 22. FIG. 26C illustrates a top view of the urethral guide 22 which is ready for insertion into a patient, wherein the balloon 42 is deflated and the meatal stop 416 is at the most proximal position at a urethral length of 50 mm. FIG. 26D illustrates another top view of the urethral guide 22, wherein the meatal stop 416 is at a 40 mm urethral position.

Referring now to FIGS. 27A and 27B, the vaginal probe body 12 comprises a distal portion 424 and a proximal portion 426. The distal portion 424 includes second and third palpation members 428, 430, each member being disposed on a side of the probe body 12 and at a centerpoint of the treatment zone 400. The second or third palpation member 428, 430 is preferably registered proximal the first palpation member 408 of the urethral guide 22 so as to position the treatment zone 400 of the probe body 12 adjacent the target tissue of the patient. Palpation members 408, 428, 430 may comprise a bump, ridge, indentation, marker, expansion member, or like mechanical palpation members.

The treatment zone 400 may have varying lengths, and generally comprises a length in a range from about 15 mm to about 30 mm. In one embodiment, the length of the treatment zone 400 along a distal-proximal axis is in a range from about 24 mm to about 26 mm. The treatment zone length from the second or third palpation member 428, 430 is thus a maximum of 13 mm for such an embodiment. As the second or third palpation member 428, 430 is registered just proximal the first palpation member 408 (which is at a fixed distance of 15 mm from the bladder neck), this geometry ensures that the treatment zone 400 will be kept away from the nerves in the area of the bladder and bladder neck as long as the patient urethral length is in excess of 32 mm. Further, the side geometry of the palpation members 428, 430 ensures that the treatment zone 400 is kept away from the urethra itself.

FIG. 27A illustrates an isometric view of the probe 12 while FIG. 27B illustrates a top view. The treatment zone 400 preferably comprises a distal electrode 18A, a center electrode 18B, and a proximal electrode 18C. These three electrodes 18A, 18B, and 18C are preferably operated in a bipolar manner. The term “treatment zone” is defined by the area of treated tissue, as for example tissue that is heated to at least 50° C. or higher for at least 50 seconds or longer. It will be appreciated that the treatment zone is smaller than the area defined by the treatment surface. For example, in the present embodiment, the three electrodes may comprise a width of 18 mm and a length of 30 mm while the treatment surface may comprise a width of 25 mm and a length of 30 mm. The width of the treatment zone is less than that of the treatment surface as the electrodes do not extend the full width of the treatment surface. This reduced width is used to ensure adequate cooling at the lateral edges of the treatment zone. This choice further insures that the treatment zone is fully within the physical dimensions of the treatment surface. Still further, this reduced width provides additional spacing between the treatment zone and adjacent nerves which run parallel to the urethra along its length. The length of the treatment zone is also less than the length of the treatment surface. As current flow is strongly biased to the shortest path between active electrode pairs, very little heating extends to the last 2 to 3 mm on the distal end of the distal electrode 18A and the last 2 to 3 mm on the proximal end of the proximal electrode 18C.

The probe body 12 further includes two visual indicators. The first indicator in the form of a marker band 432 on the probe neck provides a visual indication that the proximal electrode 18C is completely within the vagina. The surgeon may additionally lift the labia to ensure proper proximal positioning. While the length qualification and the proximal position of the probe palpation member 428, 430 relative to the guide palpation member 408 ensures that the bladder neck and bladder are protected, the physician may still move too far proximal and thus partially expose the proximal electrode 18C. Hence, the marker band 432 prevents treatment with a partially exposed proximal electrode which may lead to high current and power densities and burns. The marker band 432 further ensures the treatment zone 400 is kept away from the vaginal meatus and urethral meatus. The second indicator in the form of a reference triangle 434 on the probe neck provides an ongoing reference point prior to and during the treatment procedure so that the physician is able to assess the vaginal insertion depth of the treatment probe 12. Preferably, the reference triangle 434 will be maintained just below the guide lumen 406 so as to provide an easy visual reference point.

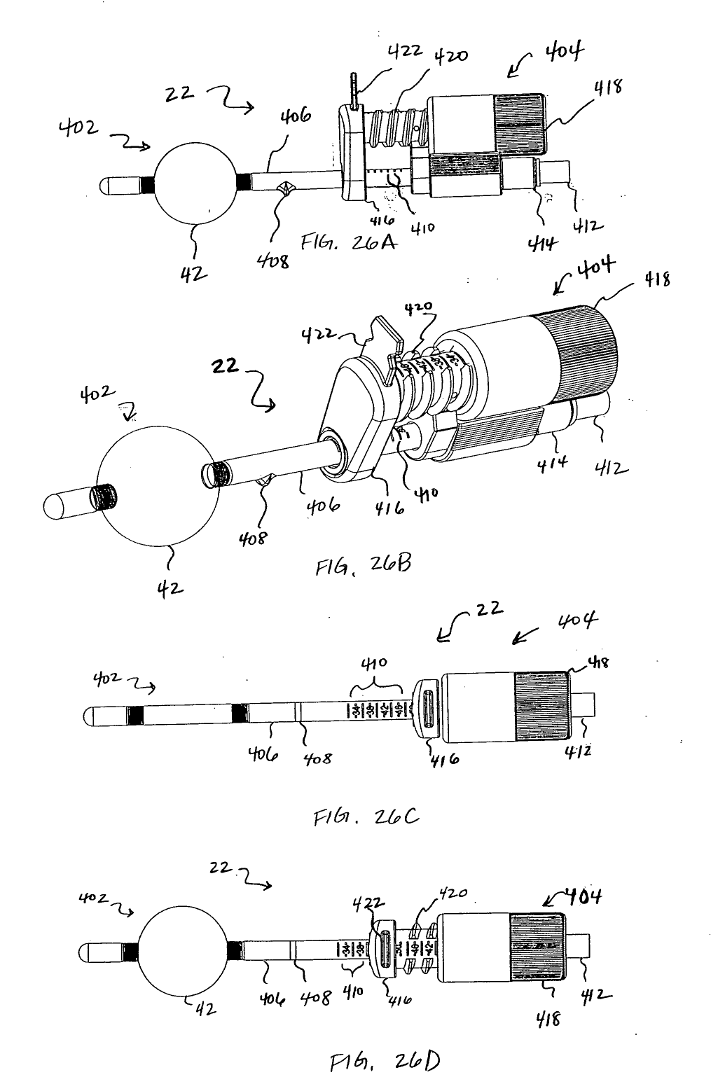

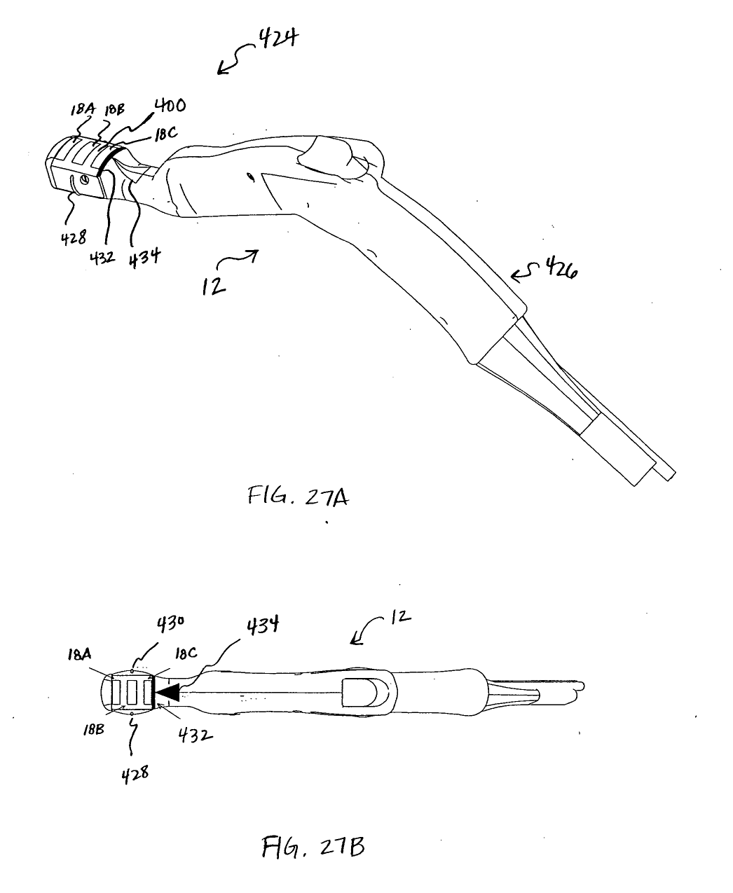

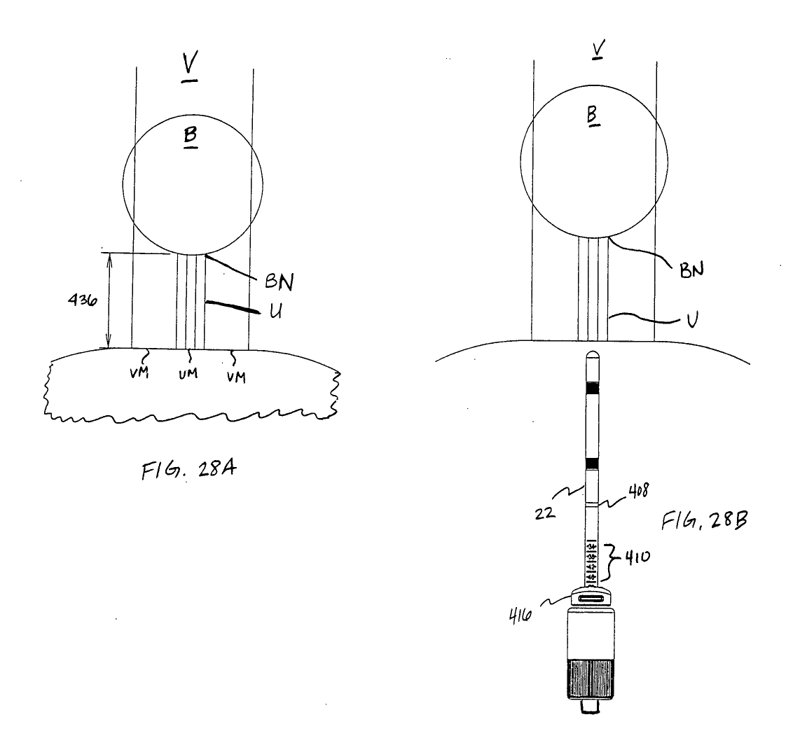

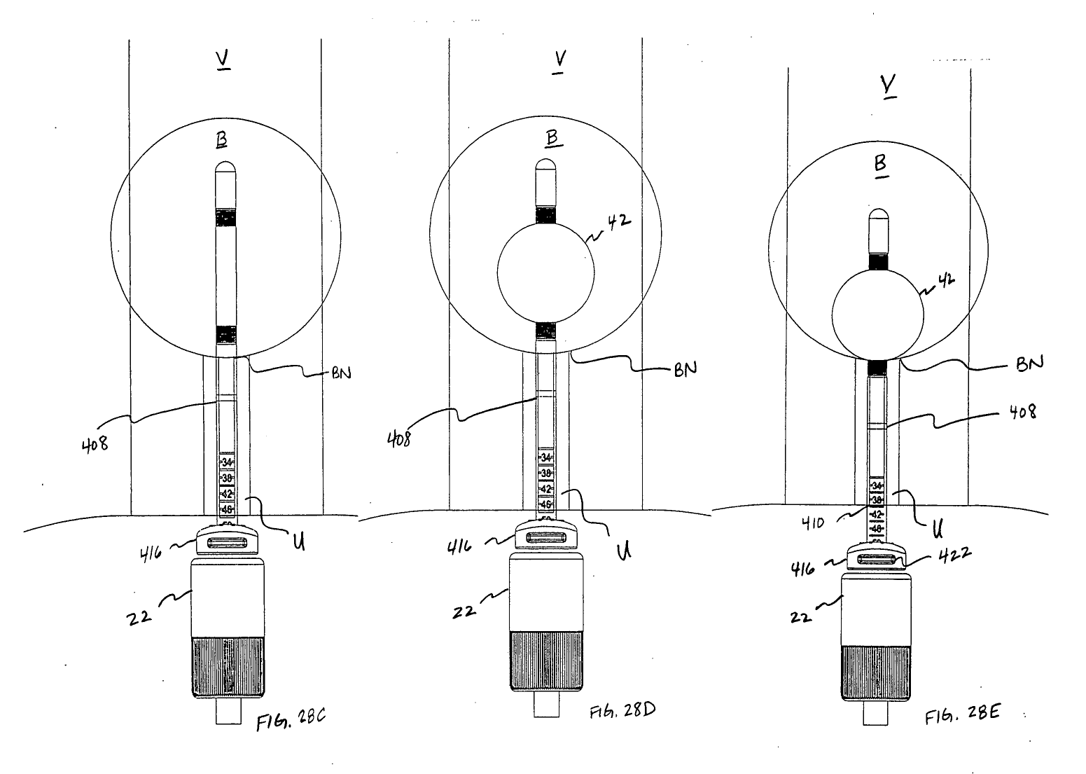

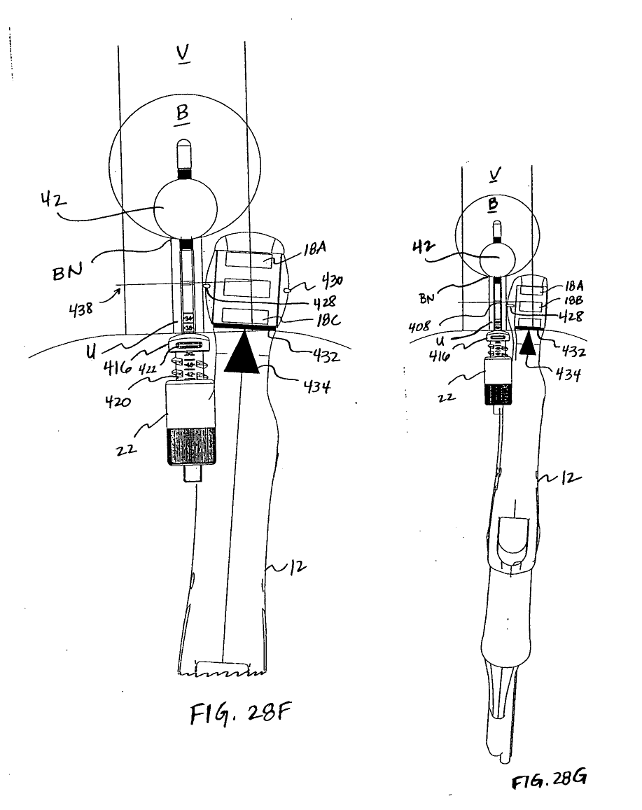

Referring now to FIGS. 28A to 28G, an exemplary palpation method for positioning the system of FIGS. 26 and 27 within a patient's body so at to direct incontinence treatment to a target tissue of the patient is illustrated. FIG. 28A depicts a top view through an abdomen of a reclining patient. The bladder B is shown superimposed on the vagina V. The bladder B is shown as if it were fully inflated. The vagina V is shown by the widely spaced axial lines. The bladder neck BN, urethra U, vaginal meatus VM, and urethral meatus UM are also illustrated. In this depiction, the urethral length is about 40 mm as indicated by arrow 436. After patient placement, the bladder B is drained with a separate catheter as already described above.

Referring now to FIG. 28B, the urethral guide 22 is adjusted to a 50 mm urethral length position as indicated by the meatal stop 416. The guide 22 is coated with a topical anesthetic gel and then inserted into the bladder B via the urethra U of the patient. FIG. 28C illustrates the distal portion 402 of the guide 22 being fully inserted into the patient's bladder B. FIG. 28D illustrates inflating the balloon 42 on the distal portion 402 of the guide 22 within the bladder B. The balloon 42 may be inflated with a variety of inflation mediums. In this example, the balloon 42 is inflated with 8 cc of sterile saline. FIG. 28E illustrates retraction of the urethral guide 22 in a proximal direction so as to seat the guide balloon 42 against the bladder neck BN. This allows for the fixed positioning of the first palpation member 408 relative to the bladder neck and the balloon 42. This fixed distance is typically about 15 mm, which is appropriate for urethral lengths of about 32 mm and higher.

The urethral length of about 40 mm is then confirmed by measurement via urethral length marker or graduation 410. As noted above, the probe 12 of the present invention is particularly well suited for urethral lengths in a range from about 32 mm to about 50 mm. As shown in FIG. 28F, the adjustment knob 418 is then rotated clockwise so as to distally advance the meatal or retention stop 416 so that it engages the urethral meatus UM. The urethral length is again confirmed to ensure that it is within the acceptable range for treatment with the probe 12 via marker 420. The abdominal portion of a retention strap is attached to the patient just above the navel while the lower portion of the retention strap is then pushed onto the attachment clip 422 on the meatal stop 416. The inflated balloon 42, retention stop 416, and retention strap ensure that the guide 22 is maintained in a stable horizontal position. Positioning may further be adjusted to hold the urethral guide 22 at a level orientation.

Prior to inserting the probe 12 into the vagina V, the physician preferably places a gloved index finger in the vagina V underneath the urethral guide 22 and palpates the first palpation member 408 on the bottom of the guide 22. The probe 12 is then inserted into the vagina V and again with the aid of the physysician's finger the probe 12 is registered with the guide 22 so as position the treatment zone 400 adjacent the target tissue of the patient as shown in FIG. 28F. In particular, the physician palpates the first palpation member 408 with the fingertip and then palpates the second palpation member 428 (for treatment of a patient's left side) on the side of the probe 12 near the first knuckle so that the probe palpation member 428 is just proximal the guide palpation member 408. As describe above in detail, the fixed positioning of the guide 22 to the bladder neck BN ensures that the probe palpation member 428, which is at a midpoint of the probe treatment zone 400, is at least 15 mm away from the bladder neck BN. As the treatment zone extends a maximum of 13 mm from the midpoint, the probe 12 is safely kept away from the nerves in the area of the bladder B and bladder neck BN as long as the patient urethral length is in excess of 32 mm. Further, the side geometry of the palpation member 428 as well as the limited electrode width relative to the treatment surface width ensures that the treatment zone 400 is kept away from the urethra U itself. The yaw denoted by line 438 is in a range of about 2 degrees to the about 6 degrees, in this instance 4 degrees.

Referring now to FIG. 28G, the probe body 12 may be rotated along its longitudinal axis in a range from about 15 degrees to about 50 degrees, in this instance the roll shown is 30 degrees. Probe 12 rotation further directs energy away from the urethra and bladder neck area. The probe 12 may additionally or alternatively be pitched upwards at an angle in a range from about 5 degrees to about 10 degrees to ensure good contact with the anterolateral wall of the vagina. Palpation positioning continues to be checked to confirm the proximal relation of the probe palpation member 428 to the guide palpation member 408. The physician may additionally lift and/or retract the labia to verify that the treatment zone 400 is completely within the vagina V and covered by the vaginal introitus via the marker band 432. Band 432 prevents treatment with a partially exposed proximal electrode 18C which may lead to high current and power densities and thus burns. The marker band 432 further ensures the treatment zone 400 is kept away from the vaginal meatus VM and urethral meatus UM. After proper positioning, the target tissue may be treated by the probe electrodes 18A, 18B, 18C with the delivery of bipolar radiofrequency energy. After treatment on the patient's left side and cool down, the same protocols as described above may be repeated with the patient's right side and the third palpation member 430.

While the above is a complete description of the preferred embodiments of the inventions, various alternatives, modifications, and equivalents may be used. For example, it may be possible to make the angular offset of the urethral guide adjustable, laterally from the probe body and/or orthogonal to a plane of the electrode. Moreover, instead of inserting the guide and probe in different body orifices, in alternative uses, both the guide and probe may be inserted in the same body orifice. Although the foregoing has been described in detail for purposes of clarity of understanding, it will be obvious that certain modifications may be practiced within the scope of the appended claim.

What is claimed is:

1. An incontinence treatment system for directing treatment to a target tissue of a patient, the system comprising:
a guide that is configured to be inserted into a urethra of the patient and positioned in a fixed relationship to an anatomical landmark; and
a probe body that is configured to be inserted into a vagina of the patient and registered relative to the guide so as to position a treatment zone of the probe adjacent the target tissue of the patient.

2. The system of claim 1, wherein the guide includes a first palpation member and the anatomical landmark comprises a bladder neck, wherein the first palpation member is positioned in a fixed relationship to the bladder neck.

3. The system of claim 2, wherein the fixed relationship comprises a distance in a range from about 12 mm to about 20 mm.

4. The system of claim 3, wherein the fixed relationship comprises a distance of about 15 mm.

5. The system of claim 2, wherein the guide includes an expandable body on a distal portion thereof which is expandable within the bladder and positionable against the bladder neck.

6. The system of claim 5, wherein the guide includes a meatus engaging surface on a proximal portion thereof which is positionable against a urethral meatus.

7. The system of claim 5, wherein the guide includes a plurality of graduations or markers on an outer lumen surface of the guide.

8. The system of claim 2, wherein the probe body includes a second palpation member on a side of the probe body and at a centerpoint of the treatment zone, wherein the second palpation member is registered proximal the first palpation member so as to position the treatment zone adjacent the target tissue.

9. The system of claim 8, wherein the treatment zone comprises a length in a range from about 15 mm to about 30 mm.

10. The system of claim 9, wherein the treatment zone comprises a length in a range from about 24 mm to about 26 mm.

11. The system of claim 8, wherein the treatment zone includes three bipolar electrodes.

12. The system of claim 8, wherein the probe body includes at least one visual indicator.

13. An incontinence treatment method for directing treatment to a target tissue of a patient, the method comprising:
inserting a guide into a urethra of the patient;
positioning the guide in a fixed relationship to an anatomical landmark;
inserting a probe into a vagina of the patient;
registering the probe with the guide so as to position a treatment zone of the probe adjacent the target tissue of the patient; and
treating the target tissue with the treatment zone.

14. The method of claim 13, wherein the guide includes a first palpation member and the anatomical landmark comprises a bladder neck, wherein positioning comprises placing the first palpation member in a fixed relationship to the bladder neck.

15. The method of claim 14, further comprising inflating a balloon in the bladder and retracting guide proximally so as to seat the balloon against the bladder neck.

16. The method of claim 15, further comprising measuring a urethral length.

17. The method of claim 15, further comprising distally advancing a retention stop so as to engage a urethral meatus.

18. The method of claim 14, wherein the probe includes a second palpation member on a side of the probe body and at a centerpoint of the treatment zone, wherein registering comprises palpating the first palpation member with the second palpation member.

19. The method of claim 18, further comprising positioning the second palpation member proximal the first palpation member.

20. The method of claim 19, further comprising verifying that the treatment zone is completely within the vagina.

21. The method of claim 19, further comprising assessing the insertion depth of the probe body in the vagina.

22. The method of claim 19, further comprising rotating the probe along its longitudinal axis in a range from about 15 degrees to about 50 degrees.

23. The method of claim 19, further comprising pitching the probe upwards at an angle in a range from about 5 degrees to about 10 degrees.

24. The method of claim 13, wherein treating comprises delivering bipolar radiofrequency energy to the target tissue with the treatment zone.
